SEQUENCE LISTING (17 sequences)

SEQ ID NO: 1 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 2 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 3 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 4 - protein, 180 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 5 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 6 - protein, 179 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 7 - protein, 117 aa, Artificial sequence
  37A10S713 heavy chain variable region
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 8 - protein, 111 aa, Artificial sequence
  37A10S713 light chain variable region
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 9 - protein, 10 aa, Artificial sequence
  37A10S713 VH CDR1
GFTFSDYWMD

SEQ ID NO: 10 - protein, 17 aa, Artificial sequence
  37A10S713 VH CDR2
NIDEDGSITEYSPFVKG

SEQ ID NO: 11 - protein, 8 aa, Artificial sequence
  37A10S713 VH CDR3
WGRFGFDS

SEQ ID NO: 12 - protein, 15 aa, Artificial sequence
  37A10S713 VL CDR1
KSSQSLLSGSFNYLT

SEQ ID NO: 13 - protein, 7 aa, Artificial sequence
  37A10S713 VL CDR2
YASTRHT

SEQ ID NO: 14 - protein, 9 aa, Artificial sequence
  37A10S713 VL CDR3
HHHYNAPPT

SEQ ID NO: 15 - protein, 447 aa, Artificial sequence
  37A10S713 heavy chain
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 218 aa, Artificial sequence
  37A10S713 light chain
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 446 aa, Artificial sequence
  37A10S713 heavy chain V2
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2022/0135682 A1
(43) Pub. Date: May 5, 2022

(54) Anti-ICOS Antibodies for the Treatment of Cancer

(71) Applicant: Jounce Therapeutics, Inc., Cambridge, MA (US)

(72) Inventor: Christopher Harvey, Boston, MA (US)

(73) Assignee: Jounce Therapeutics, Inc., Cambridge, MA (US)

(21) Appl. No.: 17/436,204

(22) PCT Filed: Mar. 10, 2020
(86) PCT No.: PCT/US2020/021821

§ 371 Date: Sep. 3, 2021

Related U.S. Application Data
(60) Provisional application No. 62/816,519, filed on Mar. 11, 2019.
(60) Provisional application No. 62/910,801, filed on Oct. 4, 2019.

(51) Int. Cl. C07K 16/28; A61P 35/00

(52) U.S. Cl. CPC C07K 16/2818; A61K 2039/505; A61P 35/00

(57) ABSTRACT

Methods of treating cancer with multiple doses of anti-ICOS antibodies and multiple doses of anti-CTLA4 antibodies are provided.

### CROSS-REFERENCE TO RELATED APPLICATIONS

This application claims the benefit of priority of U.S. Provisional Application No. 62/816,519, filed Mar. 11, 2019; and U.S. Provisional Application No. 62/910,801, filed Oct. 4, 2019, each of which is incorporated by reference herein in its entirety for any purpose.

### FIELD OF THE INVENTION

Methods of treating cancer with particular doses of anti-ICOS antibody in combination of anti-CTLA antibody are provided.

### BACKGROUND

ICOS is a member of the B7/CD28/CTLA-4 immunoglobulin superfamily and is specifically expressed on T cells. Unlike CD28, which is constitutively expressed on T cells and provides co-stimulatory signals necessary for full activation of resting T cells, ICOS is expressed only after initial T cell activation.

ICOS has been implicated in diverse aspects of T cell responses (reviewed in Simpson et al., 2010, Curr. Opin. Immunol., 22: 326-332). It plays a role in the formation of germinal centers, TB cell collaboration, and immunoglobulin class switching. ICOS-deficient mice show impaired germinal center formation and have decreased production of interleukin IL-10. These defects have been specifically linked to deficiencies in T follicular helper cells.

ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS KO mice demonstrate impaired development of autoimmune phenotypes in a variety of disease models, including diabetes (Th1), airway inflammation (Th2) and EAE neuro-inflammatory models (Th17).

In addition to its role in modulating T effector (Teff) cell function, ICOS also modulates T regulatory cells (Tregs). ICOS is expressed at high levels on Tregs, and has been implicated in Treg homeostasis and function.

Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival.

ICOS ligand (ICOSL; B7-H2; B7RP1; CD275; GL50), also a member of the B7 superfamily, is the only ligand for ICOS and is expressed on the cell surface of B cells, macrophages and dendritic cells. ICOSL functions as a non-covalently linked homodimer on the cell surface in its interaction with ICOS. Human ICOSL, although not mouse ICOSL, has been reported to bind to human CD28 and CTLA-4 (Yao et al., 2011, Immunity, 34: 729-740).

### SUMMARY

Methods of treating cancer are provided. In some embodiments, a method of treating cancer comprises administering multiple doses of an anti-ICOS agonist antibody to the subject and administering multiple doses of an anti-CTLA4 antagonist antibody, wherein each dose of the anti-ICOS agonist antibody is administered in an amount such that expected target engagement level of the anti-ICOS agonist antibody in the peripheral blood of the subject immediately prior to a subsequent administration of said anti-ICOS agonist antibody is less than about 70%, less than about 60%, less than about 50%, less than about 40%, less than about 30%, or less than about 20%. In some embodiments, the target engagement level of the anti-ICOS agonist antibody is greater than about 10%, greater than about 15%, or greater than about 20%. In certain embodiments, each dose of the anti-ICOS agonist antibody is administered in an amount such that expected target engagement level of the anti-ICOS agonist antibody in the peripheral blood of the subject immediately prior to a subsequent administration of said anti-ICOS agonist antibody is equal to or less than about 20%, equal to or less than about 10%, equal to or less than about 5%, or is 0%.

In some embodiments, a method of treating cancer in a subject comprises administering multiple doses of an anti-ICOS agonist antibody to the subject and administering multiple doses of an anti-CTLA4 antagonist antibody, wherein a dose of the anti-ICOS agonist antibody is administered once every six weeks and a dose of the CTLA4 antagonist antibody is administered once every six weeks.

In some embodiments, the first dose of the anti-ICOS agonist antibody is administered after the first dose of the anti-CTLA4 antagonist antibody. In some embodiments, the first dose of the anti-ICOS agonist antibody is administered three weeks after the first dose of the anti-CTLA4 antagonist antibody.

In some embodiments, the method comprises administering more doses of the anti-ICOS agonist antibody than the anti-CTLA4 antagonist antibody. In some embodiments, the method comprises administering four doses of the anti-CTLA4 antagonist antibody. In some embodiments, the method comprises administering at least one, at least two, at least three, at least four, or at least five doses of the anti-ICOS agonist antibody after the last dose of the anti-CTLA4 antagonist antibody has been administered.

In some embodiments, the method comprises:
1) administering a first dose of the anti-CTLA4 antagonist antibody,
2) administering a first dose of the anti-ICOS agonist antibody three weeks after the first dose of the anti-CTLA4 antagonist antibody,
3) administering a second dose of the anti-CTLA4 antagonist antibody three weeks after the first dose of the anti-ICOS agonist antibody,
4) administering a second dose of the anti-ICOS agonist antibody three weeks after the second dose of the anti-CTLA4 antagonist antibody,
5) administering a third dose of the anti-CTLA4 antagonist antibody three weeks after the second dose of the anti-ICOS agonist antibody,
6) administering a third dose of the anti-ICOS agonist antibody three weeks after the third dose of the anti-CTLA4 antagonist antibody,
7) administering a fourth dose of the anti-CTLA4 antagonist antibody three weeks after the third dose of the anti-ICOS agonist antibody, and
8) administering a fourth dose of the anti-ICOS agonist antibody three weeks after the fourth dose of the anti-CTLA4 antagonist antibody.

In some embodiments, the method further comprises administering a fifth dose of the anti-ICOS agonist antibody six weeks after the fourth dose of the anti-ICOS agonist antibody. In some embodiments, the method further comprises administering a sixth dose of the anti-ICOS agonist antibody six weeks after the fifth dose of the anti-ICOS agonist antibody.

In some embodiments, each dose of the anti-ICOS agonist antibody is 0.1 mg/kg. In some embodiments, each dose of the anti-ICOS agonist antibody is 0.03 mg/kg.

In some embodiments, the anti-CTLA4 antagonist antibody is selected from ipilimumab, tremelimumab, AGEN1181 (Agenus), AGEN1884 (Agenus), AGEN2041 (Agenus), and IBI310 (Innovent Biologics). In some embodiments, the anti-CTLA4 antagonist antibody is ipilimumab.

In some embodiments, each dose of the anti-CTLA4 antagonist antibody is 3 mg/kg.

In some embodiments, administration of the first dose of the anti-CTLA4 antagonist antibody results in the emergence or increase of an ICOShi T-cell population in the peripheral blood of the subject prior to the administration of said anti-ICOS agonist antibody.

In some embodiments, the anti-ICOS agonist antibody is selected from vopratelimab, GSK-3359069 (GSK), KY1044 (Kymab), KY1055 (Kymab), and BMS-986226 (Bristol-Myers Squibb).

In some embodiments, the anti-ICOS agonist antibody comprises an HCDR1 comprising the amino acid sequence of SEQ ID NO: 9; an HCDR2 comprising the amino acid sequence of SEQ ID NO: 10; an HCDR3 comprising the amino acid sequence of SEQ ID NO: 11; an LCDR1 comprising the amino acid sequence of SEQ ID NO: 12; an LCDR2 comprising the amino acid sequence of SEQ ID NO: 13; and an LCDR3 comprising the amino acid sequence of SEQ ID NO: 14.

In some embodiments, the anti-ICOS antibody comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 7 and the VL is at least 90%, 91%, 92%, 93%, 94%, 95%, 96%, 97%, 98%, 99%, or 100% identical to the amino acid sequence of SEQ ID NO: 8.

In some embodiments, the anti-ICOS agonist antibody comprises a VH comprising the amino acid sequence of SEQ ID NO: 7 and a VL comprises the amino acid sequence of SEQ ID NO: 8. In some embodiments, the anti-ICOS agonist antibody comprises a heavy chain comprising the amino acid sequence of SEQ ID NO: 15 and a light chain comprising the amino acid sequence of SEQ ID NO: 16. In some embodiments, the anti-ICOS agonist antibody comprises a heavy chain comprising the amino acid sequence of SEQ ID NO: 17 and a light chain comprising the amino acid sequence of SEQ ID NO: 16.

In some embodiments, wherein the subject has a cancer selected from melanoma, lung cancer, non-small cell lung cancer (NSCLC), small cell lung cancer (SCLC), urethral cancer, renal cell carcinoma (RCC) (e.g., clear cell RCC), gastric cancer, bladder cancer, endometrial cancer, MSI-H cancer of any organ, diffuse large B-cell lymphoma (DLBCL), Hodgkin's lymphoma, ovarian cancer (e.g., endometrioid ovarian cancer), head & neck squamous cell cancer (HNSCC), acute myeloid leukemia (AML), rectal cancer, refractory testicular cancer, small bowel cancer, metastatic cutaneous squamous cell cancer, cervical cancer, MSI-high colon cancer, esophageal cancer, mesothelioma, breast cancer, and triple negative breast cancer (TNBC). In some embodiments, the cancer is selected from lung cancer, non-small cell lung cancer (NSCLC), and small cell lung cancer (SCLC). In some embodiments, the cancer is urethral cancer.

In some embodiments, the subject has not previously been treated with PD-1 or PD-L1 therapy. In some embodiments, the subject has previously been treated with at least one dose or cycle of PD-1 therapy. In some embodiments, the subject showed, as a best overall response (BOR) to the PD-1 therapy, stable disease, partial response, or complete response. In some embodiments, the PD-1 therapy is PD-1 specific or PD-L1 specific. In some embodiments, the PD-1 therapy is an anti-PD-1 antibody or an anti-PD-L1 antibody.

In some embodiments, the subject undergoes surgery and/or radiation therapy in addition to administration of an anti-ICOS antibody and anti-CTLA4 antibody as described herein. In some embodiments, the subject undergoes radiation therapy in addition to administration of an anti-ICOS antibody and an anti-CTLA4 antibody as described herein.

### BRIEF DESCRIPTION OF THE DRAWINGS

FIG. 1 shows an exemplary treatment schedule for administration of multiple doses of anti-ICOS antibody and multiple doses of anti-CTLA-4 antibody.

FIG. 2 shows average tumor volume in mice treated with JTX-1011 at 0.25 mg/kg (top left panel) and 0.05 mg/kg (top right panel). The bottom panel shows changes in target engagement of ICOS as measured by free receptor in peripheral blood (% percentage compared to the initial measurement) in mice treated with JTX-1011 at 0.25 mg/kg and 0.05 mg/kg over time.

FIG. 3 shows target engagement profiles of ICOS (JTX-2011) in peripheral blood CD4+ T cells (% percentage compared to the initial measurement) in three subjects treated with JTX-1011 at 0.1 mg/kg.

FIG. 4 shows mean expression levels of intracellular cytokine (INFγ, TNFα, IL-2) in CD4⁺ICOS^{high} and CD4⁺ICOS^{low} T cell populations from three donors, measured by flow cytometry, following treatment of the T cells with JTX-2011 for 6 hours, as compared to control populations not treated with soluble JTX-2011.

### DETAILED DESCRIPTION OF SOME EMBODIMENTS

In general, the present application provides a method of treating cancer by administering multiple doses of anti-ICOS agonist antibody (e.g., JTX-2011, described below) in combination with administering multiple doses of anti-CTLA4 antagonist antibody. Specifically, in some embodiments, a dose of the anti-ICOS agonist antibody is administered once every six weeks and a dose of the anti-CTLA4 antagonist antibody is administered once every six weeks. This schedule was based, in part, on the preclinical observation that such target engagement properties appeared to correlate with efficacy. The schedule is further based, in part, on the observation that contacting an ICOS^{HIGH} population of CD4+ T cells with an anti-ICOS agonist antibody activates of the T cells; and that treatment with an anti-CTLA4 antagonist antibody induces ICOS expression on CD4+ T cells. That is, a subject may be “primed” with an anti-CTLA4 antagonist antibody, which results in an ICOS^{HIGH} population of CD4+ T cells, and then treated with an anti-ICOS agonist antibody, which activates the T cells. Accordingly, the CTLA-4 antibody is administered to the subject at a time period before (e.g., 1 week before, 2 weeks before, three weeks before, or one month before) the administration of the anti-ICOS agonist antibody (e.g., prior to the first administration of the anti-ICOS agonist antibody). Further, in some embodiments, each dose of the anti-ICOS agonist antibody is administered in an amount such that, prior to a subsequent administration of the anti-ICOS agonist antibody, the average target engagement level of the anti-ICOS antibody in peripheral blood is equal to or lower than 70%, 60%, 50%, 40%, 30%, or 20%, but greater than about 10%. In certain embodiments, each dose of the anti-ICOS agonist antibody is administered in an amount such that, prior to a subsequent administration of the anti-ICOS agonist antibody, the average target engagement level of the anti-ICOS agonist antibody in peripheral blood is equal to or less than about 20%, equal to or less than about 10%, equal to or less than about 5%, or is 0%.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

### I. Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X”.

“ICOS” and “inducible T-cell costimulatory” as used herein refer to any native ICOS that results from expression and processing of ICOS in a cell. The term includes ICOS from any vertebrate source, including mammals such as primates (e.g., humans and cynomolgus monkeys) and rodents (e.g., mice and rats), unless otherwise indicated. The term also includes naturally occurring variants of ICOS, e.g., splice variants or allelic variants. The amino acid sequence of an exemplary human ICOS precursor protein, with signal sequence (with signal sequence, amino acids 1-20) is shown in SEQ ID NO: 1. The amino acid sequence of an exemplary mature human ICOS is shown in SEQ ID NO: 2. The amino acid sequence of an exemplary mouse ICOS precursor protein, with signal sequence (with signal sequence, amino acids 1-20) is shown in SEQ ID NO: 3. The amino acid sequence of an exemplary mature mouse ICOS is shown in SEQ ID NO: 4. The amino acid sequence of an exemplary cynomolgus monkey ICOS precursor protein, with signal sequence (with signal sequence, amino acids 1-20) is shown in SEQ ID NO: 5. The amino acid sequence of an exemplary mature cynomolgus monkey ICOS is shown in SEQ ID NO: 6.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ICOS epitope is an antibody that binds this epitope with greater affinity, avidity, more readily, and/or with greater duration than it binds to other ICOS epitopes or non-ICOS epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. With the exception of CDR1 in V_{H}, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an c constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCR1, framework (FR) 2, LCD2, FR3, and LCD3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Nonlimiting exemplary light chain constant regions include λ and K. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art (such as, for example, ELISA K_{D}, KinExA, bio-layer interferometry (BLI), and/or surface plasmon resonance devices (such as a BIAcore® device), including those described herein).

The term “K_{D}”, as used herein, refers to the equilibrium dissociation constant of an antibody-antigen interaction.

In some embodiments, the “K_{D},” “K_{d},” “Kd” or “Kd value” of the antibody is measured by using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL/minute to achieve approximately 10 response units (RU) of coupled protein. Following the injection of antigen, 1 M ethanolamine is injected to block unreacted groups. For kinetics measurements, serial dilutions of polypeptide, for example, full length antibody, are injected in PBS with 0.05% TWEEN-20™ surfactant (PBST) at 25° C. at a flow rate of approximately 25 μL/min. Association rates (kₒₙ) and dissociation rates (Ur) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51:19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is ForteBio Octet® RED96 system (Pall Corporation). See, e.g., Abdiche et al., 2008, Anal. Biochem. 377: 209-277.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity. In some embodiments, biological activity of an ICOS protein includes, for example, costimulation of T cell proliferation and cytokine secretion associated with Th1 and Th2 cells; modulation of Treg cells; effects on T cell differentiation including modulation of transcription factor gene expression; induction of signaling through PI3K and AKT pathways; and mediating ADCC.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

A “functional Fc region” possesses an “effector function” of a native sequence Fc region. Exemplary “effector functions” include Fc receptor binding; C1q binding; CDC; ADCC; phagocytosis; down regulation of cell surface receptors (for example B cell receptor; BCR), etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (for example B cell receptor); and B cell activation.

“Human effector cells” are leukocytes which express one or more FcRs and perform effector functions. In some embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, for example, from blood.

“Antibody-dependent cell-mediated cytotoxicity” or “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S Pat. No. 5,500,362 or 5,821,337 or 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, for example, as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, for example, in U.S. Pat. No. 6,194,551 B1, U.S. Pat. Nos. 7,923,538, 7,994,290 and WO 1999/51642. See also, for example, Idusogie et al., J. Immunol. 164: 4178-4184 (2000).

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, for example, 0-20% binding to the FcR compared to a native sequence IgG Fc region.

The polypeptide variant which “mediates antibody-dependent cell-mediated cytotoxicity (ADCC) in the presence of human effector cells more effectively” than a parent antibody is one which in vitro or in vivo is more effective at mediating ADCC, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, or “reference tissue”, as used herein, refers to a sample, cell or tissue obtained from a source known, or believed, not to be afflicted with the disease or condition for which a method or composition of the invention is being used to identify. In some embodiments, a reference sample, reference cell or reference tissue is obtained from a healthy part of the body of the same subject or patient in whom a disease or condition is being identified using a composition or method of the invention. In some embodiments, a reference sample, reference cell or reference tissue is obtained from a healthy part of the body of one or more individuals who are not the subject or patient in whom a disease or condition is being identified using a composition or method of the invention.

A “disease” or “disorder” as used herein refers to a condition where treatment is needed and/or desired.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include lung cancer, small-cell lung cancer (SCLC), non-small cell lung cancer (NSCLC), uretheral cancer, squamous cell cancer, small-cell lung cancer, pituitary cancer, esophageal cancer, astrocytoma, soft tissue sarcoma, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma (including uterine corpus endometrial carcinoma), salivary gland carcinoma, kidney cancer, renal cancer, liver cancer, prostate cancer, vulval cancer, thyroid cancer, hepatic carcinoma, brain cancer, testis cancer, cholangiocarcinoma, gallbladder carcinoma, gastric cancer, melanoma, mesothelioma, and various types of head and neck cancer.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering an anti-ICOS antibody. “Ameliorating” also includes shortening or reduction in duration of a symptom.

In the context of cancer, the term “treating” includes any or all of: inhibiting growth of cancer cells, inhibiting replication of cancer cells, lessening of overall tumor burden and ameliorating one or more symptoms associated with the disease.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “target engagement” or “TE,” means herein a measure for the degree of occupation of ICOS by an anti-ICOS antibody. The target engagement level can be measured by availability of free-receptor using anti-ICOS antibodies, and can be determined by methods known in the art, and in some embodiments, is determined by a method disclosed herein. The target engagement level can be determined as percentage (%) relative to a control, which may be, e.g., a peripheral blood sample from the subject being treated, but before treatment with the anti-ICOS antibody. Alternatively, the control can be a level from a matched sample (e.g., a peripheral blood sample) of a healthy individual. In some embodiments, the target engagement level of an anti-ICOS antibody is measured in a peripheral blood sample (e.g., PBMCs). In some such embodiments, upon treatment with an anti-ICOS antibody, the number of ICOS receptors on the surface of T lymphocytes that are free (i.e., not bound to antibody) may be quantified. Decrease in observed available receptors may serve as an indication that anti-ICOS antibodies are binding to ICOS.

An “expected target engagement level” means herein an experimentally determined average level of target engagement exhibited in subjects after a defined amount of time following administration of a particular dose of anti-ICOS antibody. To calculate an “expected target engagement level” for a particular dosage and time, the target engagement level as measured in at least three reference subjects at the particular time after administration of the anti-ICOS antibody at the particular dose, is summed and divided by the number of reference subjects. In some embodiments, the expected target engagement level is measured in at least five reference subjects. In some embodiments, target engagement level is determined in a sample of PBMCs from one or more subjects. Target engagement may be determined by methods known in the art, and in some embodiments, is determined by a method disclosed herein. In some embodiments, a subsequent dose of anti-ICOS antibody is administered once the expected target engagement level of the anti-ICOS antibody in peripheral blood of the subject is less than about 70%, less than about 60%, less than about 50%, less than about 40%, less than about 30%, or less than about 20%. In some embodiments, the subsequent dose is administered when the expected target engagement level of the anti-ICOS agonist antibody is greater than about 10%, or greater than about 15%, or greater than about 20%. In certain embodiments, the subsequent dose is administered once the expected target engagement level of the anti-ICOS agonist antibody in peripheral blood of the subject is equal to or less than about 20%, equal to or less than about 10%, equal to or less than about 5%, or is 0%.

In some embodiments, the terms “elevated levels of ICOS,” “elevated ICOS levels,” “ICOS at an elevated level,” “ICOS^{HIGH},” and “ICOS^{hi}” refer to increased levels of ICOS in cells (e.g., CD4+ T cells) of a subject, e.g., in a peripheral blood sample of the subject, after treatment of the subject with one or more anti-cancer therapies. The increased levels can be determined relative to a control, which may be, e.g., a peripheral blood sample from the subject being treated, but either before any treatment with the one or more anti-cancer therapies, or before treatment with a second or further cycle of the one or more anti-cancer therapies. Alternatively, the control can be a level from a matched sample (e.g., a peripheral blood sample) of a healthy individual. In some embodiments, the level of ICOS is determined at the level of expressed protein, which may be detected in some embodiments using an antibody directed to an intracellular portion of ICOS. In some embodiments, the detection using such an antibody is done by use of flow cytometry. In some embodiments, an increase of at least 2-fold (e.g., at least 3-fold, 4-fold, 5-fold, 7.5-fold, 10-fold, or 15-fold) in mean fluorescence intensity (MFI), relative to a control, indicates detection of elevated ICOS levels. In some embodiments, detection of an increase in ICOS levels in at least 5% (e.g., at least 10%, 20%, 30%, 40%, 50%, 60%, 70%, 80%, or 90%) of CD4+ T cells in a peripheral blood sample indicates a subject having an ICOS^{hi} sample. In some embodiments, an increase of at least 2-fold (e.g., at least 3-fold, 4-fold, 5-fold, 7.5-fold, 10-fold, or 15-fold) in mean fluorescence intensity (MFI), relative to a control, in at least 5% (e.g., at least 10%, 20%, 30%, 40%, 50%, 60%, 70%, 80%, or 90%) of CD4+ T cells in a peripheral blood sample indicates detection of elevated ICOS levels. In some embodiments, elevated ICOS levels refer to an increase in total ICOS expression levels (e.g., mRNA levels or protein levels) in CD4+ T cells in the peripheral blood test sample of about 10%, 20%, 30%, 40%, 50%, 60%, 70%, 80%, 85%, 90%, 95%, 100%, or greater relative to a control sample. In some embodiments, elevated ICOS levels refers to an increase in total ICOS expression levels (e.g., mRNA levels or protein levels) in the CD4+ T cells in a peripheral blood sample of about at least 1.1×, 2×, 3×, 4×, 5×, 10×, 15×, 20×, 30×, 40×, 50×, 100×, 500×, 1000×, or greater relative to a control sample.

The term “control” refers to a composition known to not contain an analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (for example, analytes).

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. Unless otherwise specified, the terms “reduce”, “inhibit”, or “prevent” do not denote or require complete prevention over all time.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased on non-treated sample of a subject individual. In some examples, a reference is obtained from one or more healthy individuals who are not the subject or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at doses and for periods of time necessary, to achieve the desired therapeutic and/or prophylactic result.

A “prophylactically effective amount” refers to an amount effective, at doses and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the doses and concentrations employed and is compatible with other ingredients of the formulation. The pharmaceutically acceptable carrier is appropriate for the formulation employed.

As used herein, the term “best overall response” (BOR) is the best response recorded from the start of the study treatment until the earliest of objective progression or start of new anti-cancer therapy, taking into account any requirement for confirmation. The patient's best overall response assignment will depend on the findings of both target and non-target disease and will also take into consideration the appearance of new lesions. The best overall response is calculated via an algorithm using the assessment responses provided by an investigator over the course of a trial.

As used herein, the term “partial response” (PR) refers to at least a 30% decrease in the sum of diameters of target lesions, taking as reference the baseline sum diameters.

As used herein, the term “complete response” (CR) refers to the disappearance of all target 15 lesions with the short axes of any target lymph nodes reduced to <10 mm.

As used herein, the term “progressive disease” (PD) refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “stable disease” (SD) refers to neither sufficient shrinkage to qualify for PR nor sufficient increase to qualify for PD, taking as reference the smallest sum of diameters while on study.

A “sterile” formulation is aseptic or essentially free from living microorganisms and their spores.

A “PD-1 therapy” encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies. Nonlimiting exemplary PD-1 therapies include nivolumab (anti-PD-1 antibody; BMS-936558, MDX-1106, ONO-4538; OPDIVO®; Bristol-Myers Squibb); pidilizumab (anti-PD-1 antibody, CureTech), pembrolizumab (anti-PD-1 antibody; KEYTRUDA®, MK-3475, lambrolizumab); durvalumab (anti-PD-L1 antibody, MEDI-4736; AstraZeneca/MedImmune); RG-7446; MSB-0010718C; AMP-224; BMS-936559 (an anti-PD-L1 antibody; Bristol-Myers Squibb); AMP-514; MDX-1105; ANB-011; anti-LAG-3/PD-1; anti-PD-1 Ab (CoStim); anti-PD-1 Ab (Kadmon Pharm.); anti-PD-1 Ab (Immunovo); anti-TIM-3/PD-1 Ab (AnaptysBio); anti-PD-L1 Ab (CoStim/Novartis); atezolizumab (an anti-PD-L1 antibody, Genentech/Roche); avelumab (an anti-PD-L1 antibody, MSB0010718C, Pfizer); KD-033, PD-1 antagonist (Agenus); STI-A1010; STI-A1110; TSR-042; and other antibodies that are directed against programmed death-1 (PD-1) or programmed death ligand 1 (PD-L1).

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dose, administration, combination therapy, contraindications and/or warnings concerning the use of such therapeutic products. These are also referred to as the Full Prescribing Information for a product in the U.S.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The term “prediction” is used herein to refer to the likelihood that a subject will respond either favorably or unfavorably to a therapeutic agent or combination of therapeutic agents. In some embodiments, the prediction relates to the extent of those responses. In some embodiments, the methods of prediction described herein can be used to make treatment decisions by choosing the most appropriate treatment modalities for a particular subject.

### II. Therapeutic Methods

Methods of treating disease in a subject in need of such treatment comprising administering an anti-ICOS antibody in combination with an anti-CTLA4 antibody are provided. Nonlimiting exemplary diseases that can be treated with anti-ICOS antibodies in combination with anti-CTLA4 antibodies include, but are not limited to, cancer.

In some embodiments, a method of treating cancer in a subject comprises administering multiple doses of an anti-ICOS agonist antibody to the subject and administering multiple doses of an anti-CTLA4 antagonist antibody, wherein a dose of the anti-ICOS agonist antibody is administered once every six weeks and a dose of the CTLA4 antagonist antibody is administered once every six weeks.

In some embodiments, the first dose of the anti-ICOS agonist antibody is administered after the first dose of the anti-CTLA4 antagonist antibody. In some embodiments, the first dose of the anti-ICOS agonist antibody is administered three weeks after the first dose of the anti-CTLA4 antagonist antibody.

In some embodiments, the method comprises administering more doses of the anti-ICOS agonist antibody than the anti-CTLA4 antagonist antibody. In some embodiments, the method comprises administering four doses of the anti-CTLA4 antagonist antibody. In some embodiments, the method comprises administering at least one, at least two, at least three, at least four, or at least five doses of the anti-ICOS agonist antibody after the last dose of the anti-CTLA4 antagonist antibody has been administered.

In some embodiments, a subsequent dose of an anti-ICOS agonist antibody is administered when the expected target engagement level of the anti-ICOS antibody in the peripheral blood of the subject is less than about 70%, or less than about 60%, or less than about 50%, or less than about 40%, or less than about 30%, or less than about 20%. In some embodiments, a subsequent dose of an anti-ICOS antibody is administered when the expected target engagement level of the anti-ICOS antibody in the peripheral blood of the subject is greater than about 10%, greater than about 15%, or greater than about 20%. In certain embodiments, a subsequent dose of an anti-ICOS antibody is administered when the expected target engagement level of the anti-ICOS antibody in the peripheral blood of the subject is equal to or less than about 20%, equal to or less than about 10%, equal to or less than about 5%, or is 0%. In certain embodiments, each dose of the anti-ICOS antibody is administered in an amount such that expected target engagement level of the anti-ICOS agonist antibody in the peripheral blood of the subject immediately prior to a subsequent administration of said anti-ICOS agonist antibody is from about 0% to about 50%, from about 0% to about 40%, from about 0% to about 30%, from about 0% to about 20%, from about 0% to about 10%, from about 0% to about 5%, or from about 5% to about 50%, from about 5% to about 40%, from about 5% to about 30%, from about 5% to about 20%, from about 5% to about 10%, from about 10% to about 70%, from about 10% to about 60%, from about 10% to about 50%, from about 10% to about 40%, from about 10% to about 30%, from about 10% to about 20%, from about 15% to about 70%, from about 15% to about 60%, from about 15% to about 50%, from about 15% to about 40%, from about 15% to about 30%, from about 15% to about 20%, from about 20% to about 70%, from about 20% to about 60%, from about 20% to about 50%, from about 20% to about 40%, or from about 20% to about 30%.

In some embodiments, the expected target engagement level has been previously determined. In some such embodiments, the expected target engagement level has been calculated as the average target engagement level in a group of reference subjects (such as at least three reference subjects, or at least five reference subjects), following administration of a particular dose of anti-ICOS agonist antibody. In some embodiments, the target engagement in the peripheral blood of the reference subjects, such as on T cells, is determined at various time points following administration of the anti-ICOS antibody. The timing of the subsequent dose is then selected based on the average target engagement level in the reference subjects.

In some embodiments, administration of an anti-CTLA4 antagonist antibody results in the emergence of, or increase in, an ICOS^{hi} T cell population in the peripheral blood of the subject. Subsequent administration of the anti-ICOS agonist antibody, in some such embodiments, activates the ICOS^{hi} T cells. In some embodiments, subsequent administration of the anti-ICOS agonist antibody increases expression of IFNγ and/or TNFα in the ICOS^{hi} T cells.

In some embodiments, the treatment regimen described herein is administered to a subject that has previous received PD-1 therapy. In some such embodiments, the subject showed as a best overall response (BOR) to the PD-1 therapy, stable disease, partial response, or complete response. The PD-1 therapy may be PD-1 specific or PD-L1 specific. In various embodiments, the PD-1 therapy is an anti-PD-1 antibody or an anti-PD-L1 antibody.

Patients that can be treated as described herein are patients having a cancer. The type of cancer can be any type of cancer listed herein or otherwise known in the art. Exemplary types of cancer include, but are not limited to, lung cancer (e.g., small cell lung cancer (SCLC), non-small cell lung cancer (NSCLC)), uretheral cancer, gastric cancer, breast cancer (e.g., triple negative breast cancer (TNBC)), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC). Also see the definition of cancer, above, for additional cancer types that can be treated according to the methods of the invention.

Patients that can be treated as described herein include patients who have not previously received an anti-cancer therapy and patients who have received previous (e.g., 1, 2, 3, 4, 5, or more) doses or cycles of one or more (e.g., 1, 2, 3, 4, 5, or more) anti-cancer therapies.

Anti-ICOS antibodies can be administered alone or with other modes of anti-cancer therapies. In some embodiments, an anti-ICOS antibody is administered in conjunction with another anti-cancer agent. In some embodiments, the anti-ICOS antibody is administered with a second therapeutic method for treatment. Thus, the administration of an antibody provided herein can be in combination with another system of treatment.

In some embodiments, the other anti-cancer therapeutic agent is anti-CTLA4 antibody, and the methods include administering multiple doses of an anti-ICOS antibody to the subject and administering multiple doses of an anti-CTLA4 antibody. In some embodiments, administration of an anti-CTLA4 antagonist antibody induces ICOS expression on CD4+ T cells, and subsequent administration of an anti-ICOS agonist antibody activates the T cells (such as the ICOS^{hi} T cells).

In some embodiments, a method of treating a cancer is provided, wherein cells within a sample of the cancer express ICOS. In some such embodiments, the cancer may be considered to be ICOS-positive, or to express ICOS. Expression of ICOS may be determined by, for example, by IHC, e.g., as discussed herein. In some embodiments, a tumor is considered to express ICOS when a sample from the tumor shows 1, 2, or 3 staining of ICOS by IHC. In some embodiments, the sample from the tumor shows 2+ or 3+ staining of ICOS by IHC. In some embodiments, a tumor sample from a subject is analyzed for ICOS expression and the subject is selected for treatment with an antibody described herein if the tumor sample shows ICOS expression. In some embodiments, the subject is selected if the tumor sample shows elevated expression of ICOS.

The anti-ICOS antibody can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of an anti-ICOS antibody is administered to a subject one or more times. In some embodiments, the effective dose of an anti-ICOS antibody may be administered multiple times, including for periods of at least a month, at least two months, at least three months, at least six months, at least a year, or at least two years. In some embodiments, the timing of anti-ICOS antibody administration is determined based on the expected target engagement level, as described herein.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated.

Pharmaceutical compositions are administered in an amount effective for increasing the number of Teff cells; activating Teff cells; depleting Treg cells; and/or increasing the ratio of Teff cells to Treg cells. In some embodiments, the Treg cells are CD4+ FoxP3+ T cells. In some embodiments, the Teff cells are CD8+ T cells. In some embodiments, the Teff cells are CD4+ FoxP3− T cells and CD8+ T cells.

In some embodiments, treatment with anti-ICOS antibody results in a pharmacodynamics readout, such as up-regulation of ICOS ligand (ICOSL). In some embodiments, up-regulation of ICOSL is observed on the surface of B cells. In some embodiments, up-regulation of ICOSL is observed on the surface of granulocytes. In some embodiments, up-regulation of ICOSL is observed on the surface of neutrophils. Up-regulation of ICOSL may be observed on cells in the tumor; on cells in the spleen; on cells in peripheral blood. Up-regulation of ICOSL on the cell surface can be detected, for example, by flow cytometry. In some embodiments, soluble ICOSL is increased in the serum following treatment with anti-ICOS antibody. Soluble ICOSL can be detected by methods including, but not limited to, ELISA, MSD, and mass spectrometry.

In some embodiments, ICOS target engagement, as measured by availability of free-receptor, by anti-ICOS antibodies may be used as a pharmacodynamics readout. In some such embodiments, upon treatment by an anti-ICOS antibody, the number of ICOS receptors on the surface of T cells that are free to bind additional antibodies may be quantified. Decrease in observed available receptors may serve as an indication that anti-ICOS antibodies are binding ICOS on the surface of the cells.

The therapeutically effective amount is, in some embodiments, 0.03 mg/kg, 0.1 mg/kg or 0.3 mg/kg. In some embodiments, the therapeutically effective amount is from 0.03 mg/kg to 0.3 mg/kg. In some embodiments, the therapeutically effective amount is from 0.03 mg/kg to 0.1 mg/kg. In some embodiments, the therapeutically effective amount is between 0.1 mg/kg and 0.3 mg/kg. In some embodiments, the therapeutically effective amount is 0.1 mg/kg. In some embodiments, the therapeutically effective amount is 0.03 mg/kg. In some embodiments, the therapeutically effective amount is 0.03, 0.04, 0.05, 0.06, 0.07. 0.08, or 0.09 mg/kg.

### Combination Therapy

In some embodiments, a dose of the anti-ICOS antibody is administered every three weeks, every four weeks, every five weeks, every six weeks, every seven weeks, every eight weeks, every nine weeks, every ten weeks, every eleven weeks, or every twelve weeks. In some embodiments, a dose of the anti-ICOS antibody is administered every six weeks.

In some embodiments, the anti-ICOS antibody is administered sequentially with a second therapeutic agent. For example, administration of the two or more therapeutic agents are administered with a time separation of more than about 15, 30, or 60 minutes, 1 day, 2 days, 3 days, 1 week, 2 weeks, 3 weeks, 4 weeks, 6 weeks, or longer. In some embodiments, the anti-ICOS antibody is administered sequentially with a second therapeutic agent with a time separation of 3 weeks.

In some embodiments, a dose of the anti-ICOS antibody is administered once every six weeks and a dose of the second therapeutic agent is administered once every six weeks.

In some embodiments, the second therapeutic agent is an anti-CTLA4 antibody. In some embodiments, a dose of the anti-ICOS agonist antibody is administered once every six weeks and a dose of the CTLA4 antagonist antibody is administered once every six weeks. In some embodiments, a dose of the anti-ICOS antibody is administered sequentially with the anti-CTLA4 antibody with a time separation of 3 weeks.

In some embodiments, a method of treating cancer in a subject comprises administering multiple doses of an anti-ICOS agonist antibody to the subject and administering multiple doses of an anti-CTLA4 antagonist antibody, wherein a dose of the anti-ICOS agonist antibody is administered once every six weeks and a dose of the CTLA4 antagonist antibody is administered once every six weeks.

In some embodiments, the first dose of the anti-ICOS agonist antibody is administered after the first dose of the anti-CTLA4 antagonist antibody. In some embodiments, wherein the first dose of the anti-ICOS agonist antibody is administered three weeks after the first dose of the anti-CTLA4 antagonist antibody.

In some embodiments, the method comprises administering more doses of the anti-ICOS agonist antibody than the anti-CTLA4 antagonist antibody.

In some embodiments, the method comprises administering four doses of the anti-CTLA4 antagonist antibody. In some embodiments, the method comprises administering at least one, at least two, at least three, at least four, or at least five doses of the anti-ICOS agonist antibody after the last dose of the anti-CTLA4 antagonist antibody has been administered.

In some embodiments, the method comprises:
1) administering a first dose of the anti-CTLA4 antagonist antibody,
2) administering a first dose of the anti-ICOS agonist antibody three weeks after the first dose of the anti-CTLA4 antagonist antibody,
3) administering a second dose of the anti-CTLA4 antagonist antibody three weeks after the first dose of the anti-ICOS agonist antibody,
4) administering a second dose of the anti-ICOS agonist antibody three weeks after the second dose of the anti-CTLA4 antagonist antibody,
5) administering a third dose of the anti-CTLA4 antagonist antibody three weeks after the second dose of the anti-ICOS agonist antibody,
6) administering a third dose of the anti-ICOS agonist antibody three weeks after the third dose of the anti-CTLA4 antagonist antibody,
7) administering a fourth dose of the anti-CTLA4 antagonist antibody three weeks after the third dose of the anti-ICOS agonist antibody, and
8) administering a fourth dose of the anti-ICOS agonist antibody three weeks after the fourth dose of the anti-CTLA4 antagonist antibody.

In some embodiments, the method further comprises administering a fifth dose of the anti-ICOS agonist antibody six weeks after the fourth dose of the anti-ICOS agonist antibody. In some embodiments, the method further comprises administering a sixth dose of the anti-ICOS agonist antibody six weeks after the fifth dose of the anti-ICOS agonist antibody.

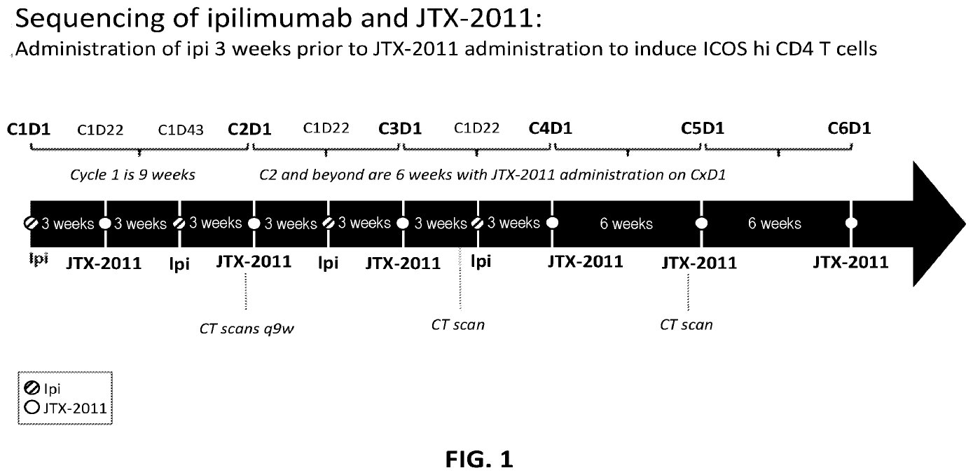

FIG. 1 shows an exemplary treatment schedule for administration of multiple doses of anti-ICOS antibody and multiple doses of anti-CTLA4 antibody.

a. Pharmaceutical Compositions

Anti-cancer therapies are administered in the practice of the methods described herein, as is known in the art (e.g., according to FDA-approved regimens) or as indicated elsewhere herein (see, e.g., above). In some embodiments, anti-cancer therapies of the invention are administered in amounts effective for treatment of cancer. The therapeutically effective amount is typically dependent on the weight of the subject being treated, his or her physical or health condition, the extensiveness of the condition to be treated, the age of the subject being treated, pharmaceutical formulation methods, and/or administration methods (e.g., administration time and administration route).

The therapeutically effective amount is, in some embodiments, 0.03 mg/kg, 0.1 mg/kg or 0.3 mg/kg body weight per dose. In some embodiments, the therapeutically effective amount is from 0.03 mg/kg to 0.3 mg/kg. In some embodiments, the therapeutically effective amount is from 0.03 mg/kg to 0.1 mg/kg. In some embodiments, the therapeutically effective amount is between 0.1 mg/kg and 0.3 mg/kg. In some embodiments, the therapeutically effective amount is 0.1 mg/kg. In some embodiments, the therapeutically effective amount is 0.03 mg/kg. In some embodiments, the therapeutically effective amount is 0.03, 0.04, 0.05, 0.06, 0.07. 0.08, or 0.09 mg/kg.

In some embodiments, each dose of the anti-ICOS antibody is 0.03 mg/kg or 0.1 mg/kg. In some embodiments, each dose of the anti-ICOS antibody is 0.1 mg/kg. In some embodiments, each dose of the anti-ICOS antibody is 0.03 mg/kg. In some embodiments, each dose of the anti-CTLA4 antibody is 3 mg/kg.

In some embodiments, compositions comprising anti-ICOS antibodies, compositions comprising anti-CTLA4 antibodies, and/or compositions comprising anti-cancer therapies are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprising an anti-ICOS antibody is provided. In some embodiments, the pharmaceutical composition comprises a humanized antibody. In some embodiments, the pharmaceutical composition comprises an antibody prepared in a host cell or cell-free system. In some embodiments, the pharmaceutical composition comprises pharmaceutically acceptable carrier.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated.

In some embodiments, anti-ICOS antibodies, anti-CTLA4 antibodies, and/or anti-cancer therapies can be administered in vivo by various routes, including, but not limited to, intravenous, intra-arterial, parenteral, intratumoral, intraperitoneal or subcutaneous. The appropriate formulation and route of administration may be selected according to the intended application.

### III. Anti-ICOS Antibodies

Antibodies directed against ICOS are provided. Anti-ICOS antibodies include, but are not limited to, humanized antibodies, chimeric antibodies, mouse antibodies, human antibodies, and antibodies comprising the heavy chain and/or light chain CDRs discussed herein. In some embodiments, an isolated antibody that binds to ICOS is provided. In some embodiments, a monoclonal antibody that binds to ICOS is provided. In some embodiments, the anti-ICOS antibody is an anti-ICOS agonist antibody. In some embodiments, administration of the anti-ICOS antibodies described herein increases the number of Teff cells and/or activates Teff cells and/or decreases Treg cells in a subject; and/or increases the ratio of Teff cells to Treg cells. In some embodiments, the Treg cells are CD4+ FoxP3+ T cells. In some embodiments, the Teff cells are CD8+ T cells. In some embodiments, the Teff cells are CD4+ FoxP3− T cells and CD8+ T cells.

Anti-ICOS antibodies described herein may be expressed and produced as described in WO 2016/154177 and WO 2017/070423, the content of each of which is incorporated by reference.

In some embodiments, the anti-ICOS antibody is an agonist antibody. See WO 2016/154177 and WO 2017/070423, which are each specifically incorporated herein by reference. Exemplary therapeutic anti-ICOS antibodies include, but are not limited to, JTX-2011 (vopratelimab, Jounce Therapeutics; US 2016/0304610; WO 2016/154177; WO 2017/070423); GSK-3359069 (GSK); KY1044 (Kymab); KY1055 (Kymab); and BMS-986226 (Bristol-Myers Squibb). In certain preferred embodiments, the agonist anti-ICOS antibody is an antibody having light and heavy chain sequences corresponding to SEQ ID NOs: 16 and 15, respectively; or SEQ ID NOs: 16 and 17, respectively). In some embodiments, the anti-ICOS antibody is JTX-2011.

In some embodiments, the anti-ICOS antibody comprises six CDRs including (a) HCDR1 comprising the amino acid sequence of SEQ ID NO: 9; (b) HCDR2 comprising the amino acid sequence of SEQ ID NO: 10; (c) HCDR3 comprising the amino acid sequence of SEQ ID NO: 11; (d) LCDR1 comprising the amino acid sequence of SEQ ID NO: 12; (e) LCDR2 comprising the amino acid sequence of SEQ ID NO: 13; and (f) LCDR3 comprising the amino acid sequence of SEQ ID NO: 14.

In some embodiments, an anti-ICOS antibody comprises a heavy chain variable region and a light chain variable region. In some embodiments, a therapeutic anti-ICOS antibody comprises at least one heavy chain comprising a heavy chain variable region and at least a portion of a heavy chain constant region, and at least one light chain comprising a light chain variable region and at least a portion of a light chain constant region. In some embodiments, a therapeutic anti-ICOS antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the anti-ICOS antibody that comprises the three heavy chain CDRs. In some embodiments, the light chain is the region of the therapeutic anti-ICOS antibody that comprises the three light chain CDRs.

In some embodiments, an anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 7. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted and/or deleted in SEQ ID NO: 7. In some embodiments, substitutions, insertions, or deletions occur in regions outside the CDRs (that is, in the FRs). Optionally, the anti-ICOS antibody comprises the VH sequence in SEQ ID NO: 7, including post-translational modifications of that sequence.

In some embodiments, the VH comprises: (a) HCDR1 comprising the amino acid sequence of SEQ ID NO: 9; (b) HCDR2 comprising the amino acid sequence of SEQ ID NO: 10; and (c) HCDR3 comprising the amino acid sequence of SEQ ID NO: 11.

In some embodiments, an anti-ICOS antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 8. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted and/or deleted in SEQ ID NO: 8. In some embodiments, the substitutions, insertions, or deletions occur in regions outside the CDRs (that is, in the FRs). Optionally, the anti-ICOS antibody comprises the VL sequence in SEQ ID 8, including post-translational modifications of that sequence.

In some embodiments, the VL comprises: (a) LCDR1 comprising the amino acid sequence of SEQ ID NO: 12; (b) LCDR2 comprising the amino acid sequence of SEQ ID NO: 13; and (c) LCDR3 comprising the amino acid sequence of SEQ ID NO: 14.

In some embodiments, an anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 7, and a light chain variable domain (VL) having at least 90%, 91%, 92%, 93%, 94%, 95%, 96%, 97%, 98%, 99%, or 100% sequence identity to the amino acid sequence of SEQ ID NO: 8. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted and/or deleted in SEQ ID NO: 7. In some embodiments, a total of 1 to 10 amino acids have been substituted, inserted and/or deleted in SEQ ID NO: 8. In some embodiments, substitutions, insertions, or deletions occur in regions outside the CDRs (that is, in the FRs). Optionally, the anti-ICOS antibody comprises the VH sequence in SEQ ID NO: 7, and the VL sequence of SEQ ID NO: 8, including post-translational modifications of one or both sequence.

In some embodiments, the anti-ICOS antibody comprises (I) a VH domain comprising: (a) HCDR1 comprising the amino acid sequence of SEQ ID NO: 9; (b) HCDR2 comprising the amino acid sequence of SEQ ID NO: 10; and (c) HCDR3 comprising the amino acid sequence of SEQ ID NO: 11; and (II) a VL domain comprising: (d) LCDR1 comprising the amino acid sequence of SEQ ID NO: 12; (e) LCDR2 comprising the amino acid sequence of SEQ ID NO: 13; and (f) LCDR3 comprising the amino acid sequence of SEQ ID NO: 14.

In some embodiments, the anti-ICOS antibody comprises the VH and VL sequences in SEQ ID NO: 7 and SEQ ID NO: 8, respectively, including post-translational modifications of those sequences.

In some embodiments, the anti-ICOS antibody binds to ICOS and increases the number of Teff cells and/or activates Teff cells and/or decreases the number of Treg cells and/or increases the ratio of Teff cells to Treg cells in a mammal, such as a human. In some embodiments, the Treg cells are CD4+ FoxP3+ T cells. In some embodiments, the Teff cells are CD8+ T cells. In some embodiments, the Teff cells are CD4+ FoxP3− T cells and/or CD8+ T cells.

### IV. ANTI-CTLA4 Antibodies

In some embodiments, an anti-ICOS antibody is administered in combination with an anti-CTLA4 antibody, such as an anti-CTLA4 antagonist antibody. An anti-CTLA-4 antagonist antibody refers to an agent capable of inhibiting the activity of cytotoxic T-lymphocyte-associated protein 4 (CTLA4), thereby activating the immune system. The CTLA-4 antibody may bind to CTLA4 and reverse CTLA-4-mediated immunosuppression. A non-limiting exemplary anti-CTLA4 antibody is ipilimumab (YERVOY®, BMS), which may be administered according to methods known in the art, e.g., as approved by the US FDA. For example, ipilimumab may be administered in the amount of 3 mg/kg intravenously over 90 minutes every three weeks for a total of 4 doses (unresectable or metastatic melanoma); or at 10 mg/kg intravenously over 90 minutes every three weeks for a total of 4 doses, followed by 10 mg/kg every 12 weeks for up to 3 years or until documented recurrence or unacceptable toxicity (adjuvant melanoma).

In some embodiments, a dose of the anti-CTLA4 antibody is administered 3 weeks before the first dose of the anti-ICOS agonist antibody. In some such embodiments, administration of the anti-CTLA4 antibody results in increased expression of ICOS on T cells, resulting in an ICOS^{hi} T cell population. Subsequent administration of the anti-ICOS agonist antibody activates the T cells, such as the ICOS^{hi} T cells. Accordingly, in various embodiments, a dose of the anti-CTLA-4 antibody is administered 3 weeks before administration of a dose of the anti-ICOS antibody for at least one, at least two, at least three, or at least four administrations.

In some embodiments, the anti-CTLA4 antibody used in the methods provided herein is ipilimumab. In some embodiments, each dose of the anti-CTLA4 antagonist antibody is 3 mg/kg. In some such embodiments, the anti-CTLA4 antibody is administered every six weeks.

Further non-limiting exemplary anti-CTLA4 antibodies include tremelimumab; AGEN1181 (Agenus); AGEN1884 (Agenus); AGEN2041 (Agenus); and IBI310 (Innovent Biologics). In some embodiments, a method comprises administering an anti-ICOS antibody in combination with tremelimumab according to a treatment schedule provided herein.

### V. Antibody Expression and Production

a. Nucleic Acid Molecules Encoding Antibodies

Nucleic acid molecules comprising polynucleotides that encode one or more chains of an antibody are provided herein. In some embodiments, a nucleic acid molecule comprises a polynucleotide that encodes a heavy chain or a light chain of an antibody. In some embodiments, a nucleic acid molecule comprises both a polynucleotide that encodes a heavy chain and a polynucleotide that encodes a light chain, of an anti-ICOS antibody. In some embodiments, a first nucleic acid molecule comprises a first polynucleotide that encodes a heavy chain and a second nucleic acid molecule comprises a second polynucleotide that encodes a light chain.

In some embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, the nucleic acid is one that encodes for any of the amino acid sequences for the antibodies in the Sequence Table herein.

Nucleic acid molecules can be constructed using recombinant DNA techniques conventional in the art. In some embodiments, a nucleic acid molecule is an expression vector that is suitable for expression in a selected host cell. Vectors

Vectors comprising polynucleotides that encode heavy chains and/or light chains are provided. Vectors comprising polynucleotides that encode heavy chains and/or light chains are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, for example, in Running Deer et al., Biotechnol. Prog. 20:880-889 (2004).

b. Host Cells

In some embodiments, antibody heavy chains and/or antibody light chains may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6° cells (Crucell); and NSO cells. In some embodiments, anti-ICOS antibody heavy chains and/or anti-ICOS antibody light chains may be expressed in yeast. See, for example, U.S. Publication No. US 2006/0270045 A1. In some embodiments, a particular eukaryotic host cell is selected based on its ability to make desired post-translational modifications to the anti-ICOS antibody heavy chains and/or anti-ICOS antibody light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the polynucleotides or vectors described herein are also provided. In some embodiments, a host cell comprising an antibody is provided. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

c. Purification of Antibodies

Antibodies can be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an antibody. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

d. Cell-Free Production of Antibodies

In some embodiments, an antibody is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

e. Antibody Compositions

In some embodiments, antibodies prepared by the methods described above are provided. In some embodiments, the antibody is prepared in a host cell. In some embodiments, the antibody is prepared in a cell-free system. In some embodiments, the antibody is purified. In some embodiments, the antibody prepared in a host cell or a cell-free system is a chimeric antibody. In some embodiments, the antibody prepared in a host cell or a cell-free system is a humanized antibody. In some embodiments, the antibody prepared in a host cell or a cell-free system is a human antibody. In some embodiments, a cell culture media comprising an antibody is provided. In some embodiments, a host cell culture fluid comprising an antibody is provided.

In some embodiments, compositions comprising antibodies prepared by the methods described above are provided. In some embodiments, the composition comprises an antibody prepared in a host cell. In some embodiments, the composition comprises an antibody prepared in a cell-free system. In some embodiments, the composition comprises a purified antibody. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

In some embodiments, a composition comprising an antibody at a concentration of more than about any one of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL is provided. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

### VI. Exemplary Methods for Detection of ICOS Expression and Target Engagement

Provided herein are methods of assessing patient responsiveness to one or more anti-cancer therapies. In some embodiments, methods of identifying a subject who may benefit from continued treatment with one or more anti-cancer therapies, optionally in combination with an anti-ICOS agonist antibody, are provided.

a. Exemplary Antibody-Based Detection Methods

In some embodiments, the methods include determining whether a patient treated with one or more anti-cancer therapies has CD4+ T cells in peripheral blood that have elevated expression of ICOS using, for example, an anti-ICOS antibody. In some embodiments, the methods of detection include contacting a patient sample (e.g., a peripheral blood sample, or a fraction thereof) with an antibody, and determining whether the level of binding differs from that of a control. In some embodiments, CD4+ T cells from the peripheral blood test sample are contacted with an anti-ICOS detection antibody and binding between the antibody and the CD4+ T cells is determined. When CD4+ T cells from a test sample are shown to have an increase in binding activity to the antibody, as compared to CD4+ T cells from a control sample, continued treatment with the one or more anti-cancer therapies is indicated, optionally in combination with anti-ICOS agonist antibody treatment, as described herein.

Various methods known in the art for detecting specific antibody-antigen binding can be used. These assays include, but are not limited to, flow cytometry (including, for example, fluorescent activating cell sorting (FACS)), indirect immune-fluorescence, solid phase enzyme-linked immunosorbent assay (ELISA), ELISpot assays, fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (MA), western blotting (including in cell western), immunofluorescent staining, microengraving (see Han et al., Lab Chip 10(11):1391-1400, 2010), Quant-iT and Qubit protein assay kits, NanoOrange protein quantitation kit, CBQCA protein quantitation kits, EZQ protein quantitation kit, Click-iT reagents, Pro-Q Diamond phosphoprotein stain, Pro-Q glycoprotein stain kits, peptide and protein sequencing, N-terminal amino acid analysis (LifeScience Technologies, Grand Island, N.Y.), chemiluminescence or colorimetric based ELISA cytokine Arrays (Signosis) Intracellular Cytokine Staining (ICS), BD Phosflow™ and BD™ Cytometric Bead Arrays (BD Sciences, San Jose, Calif.); CyTOF Mass Cytometer (DVS Sciences, Sunnyvale Calif.); Mass Spectrometry, Microplate capture and detection assay (Thermo Scientific, Rockland, Ill.), Multiplex Technologies (for example Luminex, Austin, Tex.); FlowCellect™ T-cell Activation Kit (EMD Millipore); Surface Plasmon Resonance (SPR)-based technologies (for example Biacore, GE Healthcare Life Sciences, Uppsala, Sweden); CD4+ Effector Memory T-cell Isolation Kit and CD8+CD45RA+ Effector T-cell Isolation Kit (Miltenyi Biotec Inc., CA); The EasySep™ Human T-cell Enrichment Kit (StemCells, Inc., Vancouver, Canada); HumanTh1/Th2/Th17 Phenotyping Kit (BD Biosciences, CA); immunofluorescent staining of incorporated bromodeoxyuridine (BrdU) or 7-aminoactinomycin D. See also, Current Protocols in Immunology (2004) sections 3.12.1-3.12.20 by John Wiley & Sons, Inc., or Current Protocols in Immunology (2013) or by John Wiley & Sons, Inc., the contents of which are herein incorporated by reference in their entirety.

An indicator moiety, or label group, can be attached to the subject antibodies and is selected so as to meet the needs of various uses of the method which are often dictated by the availability of assay equipment and compatible immunoassay procedures.

Appropriate labels include, without limitation, radionuclides (for example 125I, 131I, 35S, 3H, or 32P), enzymes (for example, alkaline phosphatase, horseradish peroxidase, luciferase, or β-galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

In some instances, the anti-ICOS detection antibodies need not be labeled, and the presence thereof can be detected using a second labeled antibody which binds to the first anti-ICOS antibody.

In some embodiments, the CD4+ cells from the peripheral blood test sample are contacted with an anti-ICOS detection antibody and the binding between the antibody and the CD4+ cells is determined. In some embodiments, the level of total ICOS expression in CD4+ T cells is determined using a fluorescence activated cell sorter. Fluorescence activated cell sorters can have varying degrees of sophistication, such as multiple color channels, low angle and obtuse light scattering detecting channels, impedance channels, etc. Cells may be selected against dead cells by employing dyes associated with dead cells (e.g., propidium iodide). The FACS apparatus commonly includes a light source, usually a laser, and several detectors for the detection of cell particles or subpopulations of cells in a mixture using light scatter or light emission parameters. The underlying mechanisms of FACS are well known in the art, and essentially involve scanning (e.g., counting, sorting by size or fluorescent label) single particles are they flow in a liquid medium past an excitation light source. Light is scattered and fluorescence is emitted as light from the excitation source strikes the moving particle. Forward scatter (FSC, light scattered in the forward direction, i.e., the same direction as the beam) provides basic morphological information about the particles, such as cell size and morphology. Light that is scattered at 90° to the incident beam is due to refracted or reflected light, and is referred to as side angle scatter (SSC). This parameter measures the granularity and cell surface topology of the particles. Collectively, scatter signals in both the forward and wide angle direction are used to identify subpopulations of cells based on cell size, morphology, and granularity. This information is used to distinguish various cellular populations in a heterogeneous sample.

Exemplary anti-ICOS antibodies for use in the detection aspects of the methods described herein include antibodies that recognize an internal (i.e., intracellular) epitope of ICOS. While ICOS can be expressed on the surface of T cells, it is estimated that a large proportion of total cellular ICOS (e.g., about 80%) is present in intracellular stores. While exemplary therapeutic anti-ICOS antibodies, such as JTX-2011, recognize extracellular epitopes of ICOS, the use of an anti-ICOS detection antibody that specifically binds to an intracellular ICOS epitope allows for the determination of total ICOS expression levels. Examples of antibodies that recognize intracellular ICOS epitopes, and thus which can be used in methods to detect total ICOS, include 2M13 and 2M19 (see WO 2017/070423), and variants thereof. In addition, antibodies that compete with 2M13 and 2M19 for binding to ICOS can be used to detect ICOS according to the methods of the invention.

In some embodiments, target engagement is measured following administration of an anti-ICOS antibody described herein. In some such embodiments, target engagement is measured in peripheral blood, for example, on T cells. Target engagement may be measured using, for example, an anti-ICOS antibody that binds to the same or overlapping epitope of ICOS as the administered therapeutic antibody, so the presence of the therapeutic antibody bound to ICOS blocks binding of the antibody used to measure target engagement.

In some embodiments, target engagement is measured on live CD4+ T cells in a sample taken from a subject that has been administered an anti-ICOS agonist antibody. In some such embodiments, a labeled version of the treatment antibody (such as, for example, JTX-2011-DyLight 650) is used to detect free ICOS. Receptor availability may be determined using the following formula:

$$\% Receptor Available at time t = \frac{\begin{matrix}( MFI of JTX 2011 mG 2 a - \\ mG 2 aDy 650 at time t - MFI of isotypeDy 650 at time t )\end{matrix}}{\begin{matrix}( MFI of JTX 2011 mG 2 a - \\ mG 2 aDy 650 prestudy - MFI of isotypeDy 650 prestudy )\end{matrix}} \times 100$$

Target engagement is determined as 100%−% Receptor availability.

In some embodiments, expected target engagement is determined by measuring the target engagement in a group of subjects, such as at least three subject or at least five subjects, following administration of a particular dose of anti-ICOS antibody. In some embodiments, the expected target engagement is an average of the target engagement in the group of subjects after a particularly period of time. Expected target engagement may be used to determine the appropriate dosing interval for an anti-ICOS antibody.

b. Exemplary Nucleic Acid-Based Detection Methods

In some embodiments, the methods provided herein include measuring an mRNA level. In some embodiments, the methods provided herein comprise measuring an ICOS mRNA.

Any suitable method of determining mRNA levels may be used. Methods for the evaluation of mRNAs include, for example, hybridization assays using complementary DNA probes (such as in situ hybridization using labeled riboprobes specific for target sequences, Northern blot and related techniques) and various nucleic acid amplification assays (such as RT-PCR using complementary primers specific for target sequences and other amplification type detection methods, such as, for example, branched DNA, SISB A, TMA and the like).

In some embodiments, the mRNA level is determined by quantitative RT-PCR. In some embodiments, the mRNA level is determined by digital PCR. In some embodiments, the mRNA level is determined by RNA-Seq. In some embodiments, the mRNA level is determined by RNase Protection Assay (RPA). In some embodiments, the mRNA level is determined by Northern blot. In some embodiments, the mRNA level is determined by in situ hybridization (ISH). In some embodiments, the mRNA level is determined by a method selected from quantitative RT-PCR, microarray, digital PCR, RNA-Seq, RNase Protection Assay (RPA), Northern blot, and in situ hybridization (ISH).

In some embodiments, for example when quantitative RT-PCR is used, the threshold cycle number is compared between two mRNAs, and the lower threshold indicates a higher level of the respective mRNA. As a non-limiting example, in some embodiments, if levels of ICOS mRNA and at least one reference mRNA are analyzed and the threshold cycle number (Ct) for ICOS is 28 and the Ct for the reference mRNA is 30, then ICOS is at a higher level compared to the reference. In various embodiments, similar comparisons may be carried out for any type of quantitative or semi-quantitative analytical method.

In some embodiments, the level of at least one mRNA is normalized. In some embodiments, the level of at least two mRNAs are normalized and compared to each other. In some embodiments, such normalization may allow comparison of mRNA levels when the levels are not determined simultaneously and/or in the same assay reaction. One skilled in the art can select a suitable basis for normalization, such as at least one reference mRNA or other factor, depending on the assay.

In some embodiments, the at least one reference mRNA comprises a housekeeping gene. In some embodiments, the at least one reference mRNA comprises one or more of RPLP0, PPIA, TUBB, ACTB, YMHAZ, B2M, UBC, TBP, GUSB, HPRT1, or GAPDH.

### VII. Exemplary Additional Anti-Cancer Therapies for Use in Combination with Anti-ICOS Antibodies and Anti-CTLA4 Antibodies

As examples, one or more additional anti-cancer therapies discussed herein or otherwise known in the art, can be used in connection with the methods described herein. Exemplary additional anti-cancer therapies are described below.

a. PD-1 Therapies

In some embodiments, the additional anti-cancer therapy is a PD-1 therapy. A PD-1 therapy encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies.

Exemplary PD-1 therapies include, but are not limited to, nivolumab (OPDIVO®, BMS-936558, MDX-1106, ONO-4538); pidilizumab, lambrolizumab/pembrolizumab (KEYTRUDA, MK-3475); durvalumab (anti-PD-L1 antibody, MEDI-4736; AstraZeneca/MedImmune); RG-7446; avelumab (anti-PD-L1 antibody; MSB-0010718C; Pfizer); AMP-224; BMS-936559 (anti-PD-L1 antibody); AMP-514; MDX-1105; ANB-011; anti-LAG-3/PD-1; anti-PD-1 antibody (CoStim); anti-PD-1 antibody (Kadmon Pharm.); anti-PD-1 antibody (Immunovo); anti-TIM-3/PD-1 antibody (AnaptysBio); anti-PD-L1 antibody (CoStimNovartis); RG7446/MPDL3280A (anti-PD-L1 antibody, Genentech/Roche); KD-033, PD-1 antagonist (Agenus); STI-A1010; STI-A1110; TSR-042; and other antibodies that are directed against programmed death-1 (PD-1) or programmed death ligand 1 (PD-L1).

PD-1 therapies are administered according to regimens that are known in the art, e.g., US FDA-approved regimens. In one example, nivolumab is administered as an intravenous infusion over 60 minutes in the amount of 240 mg every two weeks (unresectable or metastatic melanoma, adjuvant treatment for melanoma, non-small cell lung cancer (NSCLC), advanced renal cell carcinoma, locally advanced renal cell carcinoma, MSI-H or dMMR metastatic colorectal cancer, and hepatocellular carcinoma) or in the amount of 3 mg/kg every three weeks (classical Hodgkin lymphoma; recurrent or metastatic squamous cell carcinoma of the head and neck). In another example, pembrolizumab is administered by intravenous infusion over 30 minutes in the amount of 200 mg, once every three weeks. In another example, atezolizumab is administered by intravenous infusion over 60 minutes in the amount of 1200 mg every three weeks. In another example, avelumab is administered by intravenous infusion over 60 minutes in the amount of 10 mg/kg every two weeks. In another example, durvalumab is administered by intravenous infusion over 60 minutes in the amount of 10 mg/kg every two weeks.

b. OX40 Antibodies

In some embodiments, the additional anti-cancer therapy is an anti-OX40 agonist antibody. An OX40 agonist antibody refers to an agent that induces the activity of OX40, thereby activating the immune system and enhancing anti-tumor activity. Nonlimiting, exemplary agonist anti-OX40 antibodies include Medi6469 (MedImmune) and MOXR0916/RG7888 (Roche). These antibodies may be administered according to methods and in regimens determined to be appropriate by those of skill in the art.

c. TIGIT Antibodies

In some embodiments, the additional anti-cancer therapy is an anti-TIGIT antibody that is capable of antagonizing or inhibiting the activity of T-cell immunoreceptor with Ig and ITIM domains (TIGIT), thereby reversing TIGIT-mediated immunosuppression. Non-limiting exemplary TIGIT antibodies include OMP-313M32, BMS-986207, and the antibodies disclosed in PCT Publication Nos. WO2016028656 and WO2017053748, and U.S. Publication Nos. US20170281764 and US20160376365. These agents may be administered according to methods and in regimens determined to be appropriate by those of skill in the art.

d. IDO Inhibitors

In some embodiments, the additional anti-cancer therapy is an IDO inhibitor. An IDO inhibitor refers to an agent capable of inhibiting the activity of indoleamine 2,3-dioxygenase (IDO) and thereby reversing IDO-mediated immunosuppression. The IDO inhibitor may inhibit IDO1 and/or ID02 (INDOL1). An IDO inhibitor may be a reversible or irreversible IDO inhibitor. A reversible IDO inhibitor is a compound that reversibly inhibits IDO enzyme activity either at the catalytic site or at a non-catalytic site while an irreversible IDO inhibitor is a compound that irreversibly inhibits IDO enzyme activity by forming a covalent bond with the enzyme. Non-limiting exemplary IDO inhibitors are described, e.g., in US 2016/0060237; and US 2015/0352206. Nonlimiting exemplary IDO inhibitors include indoximod (New Link Genetics), INCB024360 (Incyte Corp), 1-methyl-D-tryptophan (New Link Genetics), and GDC-0919 (Genentech/New Link Genetics). These agents may be administered according to methods and in regimens determined to be appropriate by those of skill in the art.

e. RORγ Agonists

In some embodiments, the additional anti-cancer therapy is a RORγ agonist. RORγ agonists refer to an agent capable of inducing the activity of retinoic acid-related orphan receptor gamma (RORγ), thereby decreasing immunosuppressive mechanisms. Non-limiting exemplary RORγ agonists include, but are not limited to, LYC-55716 (Lycera/Celgene) and INV-71 (Innovimmune). These agents may be administered according to methods and in regimens determined to be appropriate by those of skill in the art.

f. Chemotherapies

In some embodiments, an additional therapeutic agent is a chemotherapeutic agent. Exemplary chemotherapeutic agents that may be combined with the anti-ICOS antibodies provided herein include, but are not limited to, capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXANE® (protein-bound paclitaxel), pemetrexed, vinorelbine, and vincristine. In some embodiments, an anti-ICOS antibody provided herein is administered with at least one kinase inhibitor. Nonlimiting exemplary kinase inhibitors include erlotinib, afatinib, gefitinib, crizotinib, dabrafenib, trametinib, vemurafenib, and cobimetanib. These agents may be administered according to methods and in regimens determined to be appropriate by those of skill in the art.

g. Cancer Vaccines

In some embodiments, an additional therapeutic agent is a cancer vaccine. Cancer vaccines have been investigated as a potential approach for antigen transfer and activation of dendritic cells. In particular, vaccination in combination with immunologic checkpoints or agonists for co-stimulatory pathways have shown evidence of overcoming tolerance and generating increased anti-tumor response. A range of cancer vaccines have been tested that employ different approaches to promoting an immune response against the tumor (see, e.g., Emens L A, Expert Opin Emerg Drugs 13(2): 295-308 (2008)). Approaches have been designed to enhance the response of B cells, T cells, or professional antigen-presenting cells against tumors. Exemplary types of cancer vaccines include, but are not limited to, peptide-based vaccines that employ targeting distinct tumor antigens, which may be delivered as peptides/proteins or as genetically-engineered DNA vectors, viruses, bacteria, or the like; and cell biology approaches, for example, for cancer vaccine development against less well-defined targets, including, but not limited to, vaccines developed from patient-derived dendritic cells, autologous tumor cells or tumor cell lysates, allogeneic tumor cells, and the like.

Exemplary cancer vaccines include, but are not limited to, dendritic cell vaccines, oncolytic viruses, tumor cell vaccines, etc. In some embodiments, such vaccines augment the anti-tumor response. Examples of cancer vaccines that can be used in combination with anti-ICOS antibodies provided herein include, but are not limited to, MAGE3 vaccine (e.g., for melanoma and bladder cancer), MUC1 vaccine (e.g., for breast cancer), EGFRv3 (such as Rindopepimut, e.g., for brain cancer, including glioblastoma multiforme), or ALVAC-CEA (e.g., for CEA+ cancers).

Nonlimiting exemplary cancer vaccines also include Sipuleucel-T, which is derived from autologous peripheral-blood mononuclear cells (PBMCs) that include antigen-presenting cells (see, e.g., Kantoff P W et al., N Engl J Med 363:411-22 (2010)). In Sipuleucel-T generation, the patient's PBMCs are activated ex vivo with PA2024, a recombinant fusion protein of prostatic acid phosphatase (a prostate antigen) and granulocyte-macrophage colony-stimulating factor (an immune-cell activator). Another approach to a candidate cancer vaccine is to generate an immune response against specific peptides mutated in tumor tissue, such as melanoma (see, e.g., Carreno B M et al., Science 348:6236 (2015)). Such mutated peptides may, in some embodiments, be referred to as neoantigens. As a nonlimiting example of the use of neoantigens in tumor vaccines, neoantigens in the tumor predicted to bind the major histocompatibility complex protein HLA-A*02:01 are identified for individual patients with a cancer, such as melanoma. Dendritic cells from the patient are matured ex vivo, then incubated with neoantigens. The activated dendritic cells are then administered to the patient. In some embodiments, following administration of the cancer vaccine, robust T-cell immunity against the neoantigen is detectable.

In some such embodiments, the cancer vaccine is developed using a neoantigen. In some embodiments, the cancer vaccine is a DNA vaccine. In some embodiments, the cancer vaccine is an engineered virus comprising a cancer antigen, such as PROSTVAC (rilimogene galvacirepvec/rilimogene glafolivec). In some embodiments, the cancer vaccine comprises engineered tumor cells, such as GVAX, which is a granulocyte-macrophage colony-stimulating factor (GM-CSF) gene-transfected tumor cell vaccine (see, e.g., Nemunaitis, 2005, Expert Rev Vaccines, 4: 259-74).

The vaccines may be administered according to methods and in regimens determined to be appropriate by those of skill in the art.

h. Additional Exemplary Anti-Cancer Therapies

Further non-limiting, exemplary anti-cancer therapies include Luspatercept (Acceleron Pharma/Celgene); Motolimod (Array BioPharma/Celgene/VentiRx Pharmaceuticals/Ligand); GI-6301 (GlobeImmune/Celgene/NantWorks); GI-6200 (GlobeImmune/Celgene/NantWorks); BLZ-945 (Celgene/Novartis); and ARRY-382 (Array BioPharma/Celgene). These and other agents may be administered according to methods and in regimens determined to be appropriate by those of skill in the art. In some embodiments, the one or more anti-cancer therapies includes surgery and/or radiation therapy. Accordingly, the anti-cancer therapies can optionally be utilized in the adjuvant or neoadjuvant setting.

In some embodiments, the additional therapeutic agent is an immune-modifying drug (IMiD). Nonlimiting exemplary IMiDs include thalidomide, lenalidomide, and pomalidomide.

In some embodiments, the additional anti-cancer therapy is a therapeutic antibody selected from cetuximab (such as ERBITUX®), elotuzumab (such as EMPLICITI®), rituximab (such as RITUXIN®), trastuzumab (such as HERCEPTIN®), and atezolizumab (such as TECENTRIQ®).

In some embodiments, the additional anti-cancer therapy is a chimeric antigen receptor T cell therapy (CAR-T therapy).

In some embodiments, the additional anti-cancer therapy is a Vascular Endothelial Growth Factor (VEGF) receptor inhibitor, such as, but not limited to, bevacizumab (Avastin®), axitinib (Inlyta®); brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); sorafenib (Nexavar®); pazopanib (Votrient®); sunitinib malate (Sutent®); cediranib (AZD2171, CAS 288383-20-1); vargatef (BIBF1120, CAS 928326-83-4); foretinib (GSK1363089); telatinib (BAY57-9352, CAS 332012-40-5); apatinib (YN968D1, CAS 811803-05-1); imatinib (Gleevec®); ponatinib (AP24534, CAS 943319-70-8); tivozanib (AV951, CAS 475108-18-0); regorafenib (BAY73-4506, CAS 755037-03-7); vatalanib dihydrochloride (PTK787, CAS 212141-51-0); brivanib (BMS-540215, CAS 649735-46-6); vandetanib (Caprelsa® or AZD6474); motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); dovitinib dilactic acid (TKI258, CAS 852433-84-2); linfanib (ABT869, CAS 796967-16-3); cabozantinib (XL184, CAS 849217-68-1); lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-β-pyridinyl)-1H-pyrazolo[3,4-d]pyrimidin-4-yl]amino]-N-[3-(trifluoromethyl)phenyl]-benzamide (BHG712, CAS 940310-85-0); and aflibercept (Eylea®).

In some embodiments, the additional anti-cancer therapy is a cytokine therapy, such as in combination with one, two, three or more cytokines. In some embodiments, the cytokine is one, two, three or more interleukins (ILs) chosen from IL-1, IL-2, IL-12, IL-15 or IL-21.

In some embodiments, the additional anti-cancer therapy is a cytokine therapy in combination with an agent that targets PTEN. Without intending to be bound by any particular theory, it is believed that enhanced PI3K signaling reduces Treg function.

In some embodiments, the additional anti-cancer therapy is an A2A receptor antagonist. In some embodiments, the A2aR antagonist is an A2aR pathway antagonist (e.g., a CD-73 inhibitor, such as an anti-CD73 antibody). A nonlimiting exemplary anti-CD73 antibody is MEDI9447. Without intending to be bound by any particular theory, targeting the extracellular production of adenosine by CD73 may reduce the immunosuppressive effects of adenosine. MEDI9447 has been reported to have a range of activities, including, for example, inhibition of CD73 ectonucleotidase activity, relief from AMP-mediated lymphocyte suppression, and inhibition of syngeneic tumor growth. In some embodiments, an anti-ICOS antibody provided herein is administered in combination with one or more of the following: i) an agonist of Stimulator of Interferon Genes (a STING agonist), (ii) an agonist of a Toll-Like Receptor (TLR) (such as an agonist of TLR-3, -4, -5, -7, -8, or -9), (iii) a TIM-3 modulator (such as an anti-TIM-3 antibody), (iv) a VEGF receptor inhibitor, (v) a c-Met inhibitor, (vi) a TGFβ inhibitor (such as an anti-TGFβ antibody), (vii) an A2AR antagonist, and/or a (viii) BTK inhibitor.

In some embodiments, an oncolytic virus is a recombinant oncolytic virus, such as those described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1. In some embodiments, a recombinant oncolytic virus, such as oncolytic NDV, comprises a nucleic acid sequence encoding a pro-apoptotic protein (such as apoptin), a cytokine (such as GM-CSF, CSF, interferon-gamma, interleukin-2 (IL-2), or tumor necrosis factor-alpha), an immunoglobulin (such as an antibody against ED-B firbonectin), a tumor associated antigen, a bispecific adapter protein (such as a bispecific antibody or antibody fragment directed against NDV HN protein and a T cell co-stimulatory receptor, such as CD3 or CD28; or a fusion protein between human IL-2 and a single chain antibody directed against NDV HN protein). See, e.g., Zamarin et al. Future Microbiol. 7.3(2012):347-67, which is incorporated herein by reference in its entirety. In some embodiments, the oncolytic virus is a chimeric oncolytic NDV, e.g., as described in U.S. Pat. No. 8,591,881 B2, US 2012/0122185 A1, and/or US 2014/0271677 A1, each of which is incorporated herein by reference in its entirety.

In some embodiments, an oncolytic virus comprises a conditionally replicative adenovirus (CRAd), which is designed to replicate exclusively in cancer cells. See, e.g., Alemany et al. Nature Biotechnol. 18(2000):723-27, which is incorporated herein by reference in its entirety. In some embodiments, an oncolytic adenovirus comprises one described in Table 1 on page 725 of Alemany et al.

Exemplary oncolytic viruses include but are not limited to the following:
Group B Oncolytic Adenovirus (ColoAdl) (PsiOxus Therapeutics Ltd.) (see, e.g., Clinical Trial Identifier: NCT02053220);
ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129);
VCN-01, which is a genetically modified oncolytic human adenovirus encoding human PH20 hyaluronidase (VCN Biosciences, S.L.) (see, e.g., Clinical Trial Identifiers: NCT02045602 and NCT02045589);
Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Catala d'Oncologia) (see, e.g., Clinical Trial Identifier: NCT01864759);
Celyvir, which comprises bone marrow-derived autologous mesenchymal stem cells (MSCs) infected with ICOVIR5, an oncolytic adenovirus (Hospital Infantil Universitario Nino Jesus, Madrid, Spain/Ramon Alemany) (see, e.g., Clinical Trial Identifier: NCT01844661); and
CG0070, which is a conditionally replicating oncolytic serotype 5 adenovirus (Ad5) in which human E2F-1 promoter drives expression of the essential Ela viral genes, thereby restricting viral replication and cytotoxicity to Rb pathway-defective tumor cells (Cold Genesys, Inc.) (see, e.g., Clinical Trial Identifier: NCT02143804); or DNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

Exemplary BTK inhibitors include, but are not limited to, ibrutinib (PCI-32765); GDC-0834; RN-486; CGI-560; CGI-1764; HM-71224; CC-292; ONO-4059; CNX-774; or LFM-A13. In some embodiments, a BTK inhibitor does not reduce or inhibit the kinase activity of interleukin-2-inducible kinase (ITK). In some such embodiments, the BTK inhibitor is selected from GDC-0834; RN-486; CGI-560; CGI-1764; HM-71224; CC-292; ONO-4059; CNX-774; or LFM-A13. In some embodiments, a kinase inhibitor is a BTK inhibitor, such as ibrutinib (PCI-32765).

In some embodiments, the additional anti-cancer therapy is an IL-33 and/or IL-33R inhibitors (such as, for example, an anti-IL-33 antibody or an anti-IL-33R antibody).

In some embodiments, the additional anti-cancer therapy is an acyl coenzyme A-cholesterol acyltransferase (ACAT) inhibitor, such as avasimibe (CI-1011).

In some embodiments, the additional anti-cancer therapy is an inhibitor of chemokine (C-X-C motif) receptor 2 (CXCR2). In some embodiments, the CXCR2 inhibitor is danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea, and is described, e.g., in Miller et al. Eur J Drug Metab Pharmacokinet (2014) 39:173-181; and Miller et al. BMC Pharmacology and Toxicology (2015), 16:18. In some embodiments, the CXCR2 inhibitor is reparixin (CAS Registry Number: 266359-83-5). Reparixin is also known as repertaxin or (2R)-2-[4-(2-methylpropyl)phenyl]-N-methylsulfonylpropanamide, and is a non-competitive allosteric inhibitor of CXCR1/2. Reparixin is described, e.g., in Zarbock et al. British Journal of Pharmacology (2008), 1-8. In some embodiments, the CXCR2 inhibitor is navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]aminoThenzamide, and is described, e.g., in Ning et al. Mol Cancer Ther. 2012; 11(6):1353-64.

In some embodiments, the additional anti-cancer therapy is a CD27 agonist. In some embodiments, the CD27 agonist is varlilumab (CAS Registry Number: 1393344-72-3). Varlilumab is also known as CDX-1127 (Celldex) or 1F5, and is a fully human monoclonal antibody that targets CD27. Varlilumab activates human T cells in the context of T cell receptor stimulation and therefore mediates anti-tumor effects. Varlilumab also provides direct therapeutic effects against tumors that express CD27. Varlilumab is described, e.g., in Vitale et al., Clin Cancer Res. 2012; 18(14):3812-21, WO 2008/051424, and U.S. Pat. No. 8,481,029. In some embodiments, the CD27 agonist is BION-1402 (BioNovion), which is also known as hCD27.15. BION-1402 is an anti-human CD27 monoclonal antibody that stimulates the proliferation and/or survival of CD27+ cells. BION-1402 activates human CD27 more effectively than its ligand CD70, which results in a significantly increased effect on proliferation of CD8+ and CD4+ T-cells. BION-1402 is disclosed, e.g., as hCD27.15 in WO 2012/004367. The antibody is produced by hybridoma hCD27.15, which was deposited with the ATCC in on Jun. 2, 2010 under number PTA-11008.

### VIII. Examples

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Mouse Syngeneic Tumor Model

### Study Design

In the mouse experiments described herein, anti-ICOS antibody JTX-1011-mG2a was used, which is a chimeric version of JTX-2011 having a murine IgG2a constant region.

Six to eight week old female A/J mice were inoculated via a subcutaneous (s.c.) injection on the right flank with 1×10⁶ Sal/N cells in 100 ml PBS using tuberculin syringes with 27-gauge needles. Tumor growth was monitored and on day 7, animals were redistributed into new cages after normalizing the average tumor volume to 100 mm³ for each treatment group. Ten mice were included in each treatment group. Animals were administered 0.05 mg/kg or 0.25 mg/kg antibody (JTX-1011-mG2a or isotype control) via intraperitoneal (i.p.) injection once weekly for two weeks. Tumor volumes were measured twice weekly.

Additional Sal/N tumor bearing mice were injected with JTX-1011-mG2a for assessment of circulating JTX-1011-mG2a levels. A/J mice were inoculated subcutaneously with Sal/N cells on day −7. On day 0, mice were i.p. injected with JTX-1011-mG2a at 0.25 or 0.05 mg/kg.

To assess target engagement (based on receptor availability), blood was collected in EDTA coated microtubes via submandibular draw for serial bleeds and cardiac puncture for terminal bleeds. The following time points were collected for receptor availability analysis by flow cytometry: 0, 6, 24, 48, 72, 96, 120, and 168 hrs. The first three time points were serial bleeds from the same set of mice, the next three time points were serial bleeds from a second set of mice, and the final two time points were serial bleeds from a third set of mice. Terminal time points were at 24, 96 and 168 hrs, and tumor tissue was taken and analyzed at those time points.

CD4+ T cells were identified as live CD3+CD4+. A labeled version of the treatment antibody (JTX-2011-DyLight 650) was used to detect free ICOS. Receptor availability was determined using the following formula:

$$\% Receptor Available at time t = \frac{\begin{matrix}( MFI of JTX 2011 mG 2 a - \\ mG 2 aDy 650 at time t - MFI of isotypeDy 650 at time t )\end{matrix}}{\begin{matrix}( MFI of JTX 2011 mG 2 a - \\ mG 2 aDy 650 prestudy - MFI of isotypeDy 650 prestudy )\end{matrix}} \times 100$$

Target engagement was determined as 100%−% Receptor availability.

### Results

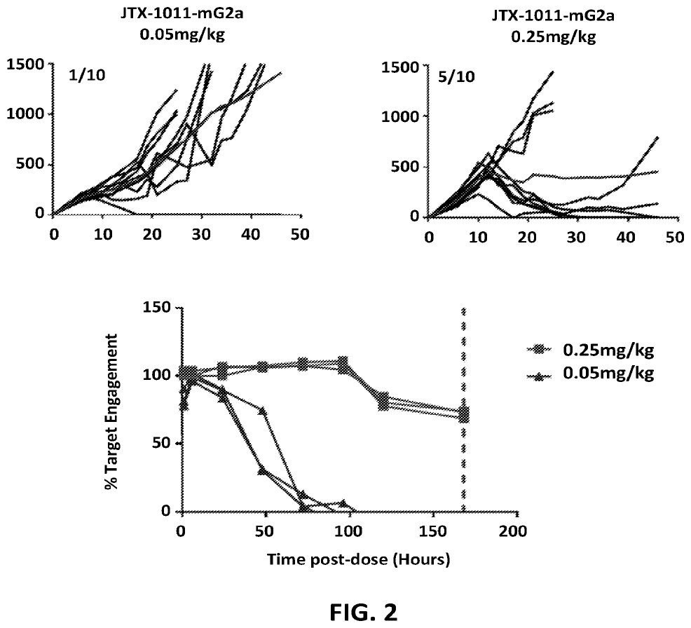

Assessment of Tumor Volume. As shown in FIGS. 2A-2B, in mice treated with 0.05 mg/kg of JTX-1011-mG2a, only 1 out of 10 tumors showed complete regression. In the mice treated with 0.25 mg/kg of JTX-1011-mG2a, 5 of 10 tumors showed complete regression, and one additional animal in the 0.25 mg/kg had stable disease with the tumor stabilizing at about 400 mm³.

Target engagement on peripheral blood CD4 T cells. As shown in FIG. 2C, in the peripheral blood, ICOS availability decreased on total CD4 T cells following i.p. administration of JTX-1011-mG2a. After an initial dose of 0.25 mg/kg, target engagement was 100% and did not begin to decrease until after 100 hr post dosing. At the time of the second dose administration (dashed line), the target engagement was still greater than 50%. In contrast, after an initial dose at 0.05 mg/kg, target engagement was approximately 90%, and began to decrease by 24 hr post dosing. At the time of the second dose administration, no target engagement detected (˜0%).

Without intending to be bound by any particular theory, it is believed that when target engagement falls below about 70%, sufficient target is available for the subsequent dose of JTX-1011-mG2a to be effective; that is, target engagement need not reach 0% before administration of the subsequent dose. As shown in FIG. 2B, the mice group with the 0.25 mg/kg JTX-1011-mG2a that showed about 50% target engagement at the time of the second dose administration, resulted in tumor regression or tumor stabilization at a higher rate than the mice administered 0.05 mg/kg JTX-1011-mG2a, in which target engagement reached 0% before the second dose.

### Example 2. Target Engagement Profile of JTX-2011

The appropriate timing of the second dose of JTX-2011 was determined based on a clinical study of human subjects treated with JTX-2011. JTX-2011 was administered to subjects by a 1 hr intravenous (i.v.) infusion at a dose of 0.1 mg/kg. Blood was obtained on Day 1 (pre-dose), Day 2 (24 hours post-Day 1 dose), Day 7, Day 14, Day 21, and Day 42.

Samples of whole blood were first Fc blocked with 5 μL Human TruStain (Biolegend) for 15 min on ice. Following Fc block, 100 μL of an antibody mix containing anti-CD3 FITC, anti-CD4 PE/Cy7, viability dye e780, and JTX-2011-DyLight 650 (or anti-RSV DyLight 650 as an isotype control). Blood/antibody mix was incubated on ice for 45 min. Following incubation, samples were centrifuged at 500×g for 5 min. Supernatant was decanted, and samples were resuspended in 200 μL of FACS staining buffer. Wash steps were repeated three times, with final resuspension in 200 μL staining buffer+0.1% paraformaldehyde.

ICOS target engagement (as assessed by receptor availability substantially as described above) was assessed on subjects' peripheral blood CD4+ T cells by flow cytometry (BD Fortessa flow cytometer).

### Results

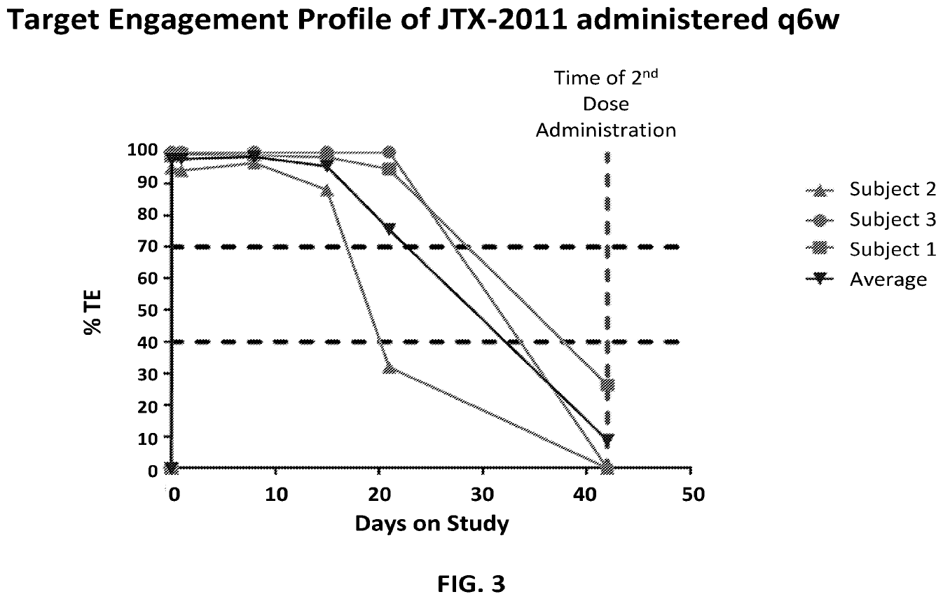

As shown in FIG. 3, After an initial dose of 0.1 mg/kg, average target engagement was found to be 100% and did not begin to decrease until after Day 14 (post first dose). At Day 42, the target engagement in all three subjects was under 40%, with an average target engagement of about 10%. Based on this data, the second dose administration at Day 42 (6 weeks from the first dose) gives a desired profile of peripheral target engagement.

### Example 3. Examination of Polyfunctional Cytokine Responses in ICOS' Cells with and without JTX-2011 Treatment

Peripheral blood mononuclear cells (PBMCs) from three healthy donors were stimulated with soluble recall antigen (tetanus antigen) for 24 hours to induce differentiated ICOS^{hi} and ICOS^{low} CD4 T cell populations (in ICOS^{hi}CD4⁺ and ICOS^{lo}CD4⁺ populations). Following stimulation, soluble JTX-2011 or isotype control antibody were added to the PBMC solution along with brefeldin A and incubated for 6 hours. Following the 6-hour ex vivo treatment, expression of intracellular cytokine (INFγ, TNFα, IL-2) was measured by flow cytometry in CD4⁺ICOS^{hi} and CD4⁺ICOS^{lo} populations. Mean levels of cytokines IFN-γ, TNFα, and IL-6 were determined and compared to control populations not treated with soluble JTX-2011.

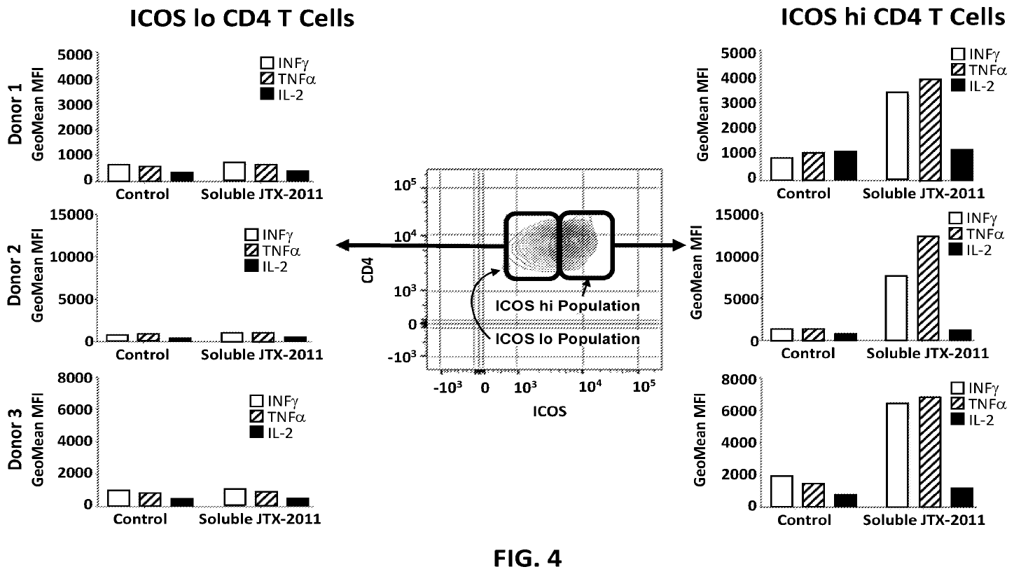

As shown in FIG. 4, in the ICOS^{hi} CD4⁺ populations, a significant increase in INFγ and TNFα expression levels were observed following treatment with JTX-2011 as compared to treatment with control antibody; whereas in the ICOS¹⁰ CD4⁺ populations, cytokine expression did not increase following treatment with either antibody. These results demonstrate that treatment of ICOS^{hi} CD4⁺ T cells with JTX-2011 further enhances INFγ and TNFα expression, indicating activation of T cells.

### Example 4. Design of Combination Therapy Regimen

FIG. 1 shows an exemplary treatment schedule for administration of multiple doses of anti-ICOS antibody (JTX-2011) and multiple doses of anti-CTLA-4 antibody (e.g., ipilimumab).

3 mg/kg ipilimumab is administered once every six weeks four times, and JTX-2011 is administered once every six weeks for at least four times.

The first dose of ipilimumab is administered at the beginning of the treatment cycle. Ipilimumab is expected to increase the population of ICOS^{hi}CD4⁺ T cells in the subject. The first dose of JTX-2011 is administered three weeks after the first dose of ipilimumab. As shown herein, stimulation of ICOS^{hi} CD4⁺ T cells with JTX-2011 activates the T cells. A second dose of ipilimumab is then administered three weeks after the first dose of JTX-2011, and a second dose of JTX-2011 is administered three weeks after the second dose of ipilimumab. A third dose of ipilimumab is administered three weeks after the second dose of JTX-2011, and a third dose of JTX-2011 is administered three weeks after the third dose of ipilimumab. A fourth dose of ipilimumab is administered three weeks after the third dose of JTX-2011, and a fourth dose of JTX-2011 is administered three weeks after the fourth dose of ipilimumab. JTX-2011 is then administered again six weeks after the fourth dose of JTX-2011, and then again after another six weeks.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Sequences

| SEQ | | |
|---|---|---|
| ID | | |
| NO | Description | Sequence |
| 1 | Human ICOS precursor | MKSGLWYFFL FCLRIKVLTG EINGSANYEM FIFHNGGVQI |
| | (with signal sequence); | LCKYPDIVQQ FKMQLLKGGQ ILCDLTKTKG SGNTVSIKSL |
| | UniProtKB/Swiss-Prot: | KFCHSQLSNN SVSFFLYNLD HSHANYYFCN LSIFDPPPFK |
| | Q9Y6W8.1; 7 Jan. 2015 | VTLTGGYLHI YESQLCCQLK FWLPIGCAAF VVVCILGCIL |
| | | ICWLTKKKYS SSVHDPNGEY MFMRAVNTAK KSRLTDVTL |
| 2 | Human mature ICOS | EINGSANYEM FIFHNGGVQI LCKYPDIVQQ FKMQLLKGGQ |
| | (without signal sequence) | ILCDLTKTKG SGNTVSIKSL KFCHSQLSNN SVSFFLYNLD |
| | | HSHANYYFCN LSIFDPPPFK VTLTGGYLHI YESQLCCQLK |
| | | FWLPIGCAAF VVVCILGCIL ICWLTKKKYS SSVHDPNGEY |
| | | MFMRAVNTAK KSRLTDVTL |
| 3 | Mouse ICOS precursor | MKPYFCRVFV FCFLIRLLTG EINGSADHRM FSFHNGGVQI |
| | (with signal sequence); | SCKYPETVQQ LKMRLFRERE VLCELTKTKG SGNAVSIKNP |
| | UniProtKB/Swiss-Prot: | MLCLYHLSNN SVSFFLNNPD SSQGSYYFCS LSIFDPPPFQ |
| | Q9WVS0.2; 7 Jan. 2015 | ERNLSGGYLH IYESQLCCQL KLWLPVGCAA FVVVLLFGCI |
| | | LIIWFSKKKY GSSVHDPNSE YMFMAAVNTN KKSRLAGVTS |
| 4 | Mouse mature ICOS | EINGSADHRM FSFHNGGVQI SCKYPETVQQ LKMRLFRERE |
| | (without signal sequence) | VLCELTKTKG SGNAVSIKNP MLCLYHLSNN SVSFFLNNPD |
| | | SSQGSYYFCS LSIFDPPPFQ ERNLSGGYLH IYESQLCCQL |
| | | KLWLPVGCAA FVVVLLFGCI LIIWFSKKKY GSSVHDPNSE |
| | | YMFMAAVNTN KKSRLAGVTS |
| 5 | Cynomolgus monkey ICOS | MKSGLWYFFL FCLHMKVLTG EINGSANYEM FIFHNGGVQI |
| | precursor (with signal | LCKYPDIVQQ FKMQLLKGGQ ILCDLTKTKG SGNKVSIKSL |
| | sequence) | KFCHSQLSNN SVSFFLYNLD RSHANYYFCN LSIFDPPPFK |
| | | VTLTGGYLHI YESQLCCQLK FWLPIGCATF VVVCIFGCIL |
| | | ICWLTKKKYS STVHDPNGEY MFMRAVNTAK KSRLTGTTP |
| 6 | Cynomolgus mature ICOS | EINGSANYEM FIFHNGGVQI LCKYPDIVQQ FKMQLLKGGQ |
| | (without signal sequence) | ILCDLTKTKG SGNKVSIKSL KFCHSQLSNN SVSFFLYNLD |
| | | RSHANYYFCN LSIFDPPPFK VTLTGGYLHI YESQLCCQLK |
| | | FWLPIGCATF VVVCIFGCIL ICWLTKKKYS STVHDPNGEY |
| | | MFMRAVNTAK KSRLTGTTP |
| 7 | 37A10S713 heavy chain | EVQLVESGGG LVQPGGSLRL SCAASGFTFS DYWMDWVRQA |
| | variable region | PGKGLVWVSN IDEDGSITEY SPFVKGRFTI SRDNAKNTLY |
| | | LQMNSLRAED TAVYYCTRWG RFGFDSWGQG TLVTVSS |
| 8 | 37A10S713 light chain | DIVMTQSPDS LAVSLGERAT INCKSSQSLL SGSFNYLTWY |
| | variable region | QQKPGQPPKL LIFYASTRHT GVPDRFSGSG SGTDFTLTIS |
| | | SLQAEDVAVY YCHHHYNAPP TFGPGTKVDI K |
| 9 | 37A10S713 VH CDR1 | GFTFSDYWMD |
| 10 | 37A10S713 VH CDR2 | NIDEDGSITEYSPFVKG |
| 11 | 37A10S713 VH CDR3 | WGRFGFDS |
| 12 | 37A10S713 VL CDR1 | KSSQSLLSGSFNYLT |
| 13 | 37A10S713 VL CDR2 | YASTRHT |
| 14 | 37A10S713 VL CDR3 | HHHYNAPPT |
| 15 | 37A10S713 human IgG1 | EVQLVESGGG LVQPGGSLRL SCAASGFTFS DYWMDWVRQA |
| | heavy chain | PGKGLVWVSN IDEDGSITEY SPFVKGRFTI SRDNAKNTLY |
| | | LQMNSLRAED TAVYYCTRWG RFGFDSWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APELLGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPGK |
| 16 | 37A10S713 human κ light | DIVMTQSPDS LAVSLGERAT INCKSSQSLL SGSFNYLTWY |
| | chain | QQKPGQPPKL LIFYASTRHT GVPDRFSGSG SGTDFTLTIS |
| | | SLQAEDVAVY YCHHHYNAPP TFGPGTKVDI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| 17 | 37A10S713 human IgG1 | EVQLVESGGG LVQPGGSLRL SCAASGFTFS DYWMDWVRQA |
| | heavy chain V2 | PGKGLVWVSN IDEDGSITEY SPFVKGRFTI SRDNAKNTLY |
| | | LQMNSLRAED TAVYYCTRWG RFGFDSWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APELLGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPG |

What is claimed is:

1. A method of treating cancer in a subject, comprising administering multiple doses of an anti-ICOS agonist antibody to the subject and administering multiple doses of an anti-CTLA4 antagonist antibody, wherein each dose of the anti-ICOS agonist antibody is administered in an amount such that the expected target engagement level of the anti-ICOS agonist antibody in the peripheral blood of the subject immediately prior to a subsequent administration of said anti-ICOS agonist antibody is less than about 70%, less than about 60%, less than about 50%, less than about 40%, less than about 30%, or less than about 20%.

2. The method of claim 1, wherein the expected target engagement level of the anti-ICOS agonist antibody in the peripheral blood of the subject immediately prior to a subsequent administration of said anti-ICOS agonist antibody is greater than about 10%, greater than about 15%, or greater than about 20%.

3. The method of claim 1, wherein the expected target engagement level of the anti-ICOS agonist antibody in the peripheral blood of the subject immediately prior to a subsequent administration of said anti-ICOS agonist antibody is equal to or less than about 20%, equal to or less than about 10%, equal to or less than about 5%, or is 0%.

4. A method of treating cancer in a subject, comprising administering multiple doses of an anti-ICOS agonist antibody to the subject and administering multiple doses of an anti-CTLA4 antagonist antibody, wherein a dose of the anti-ICOS agonist antibody is administered once every six weeks and a dose of the CTLA4 antagonist antibody is administered once every six weeks.

5. The method of any one of claims 1-4, wherein the first dose of the anti-ICOS agonist antibody is administered after the first dose of the anti-CTLA4 antagonist antibody.

6. The method of claim 5, wherein the first dose of the anti-ICOS agonist antibody is administered three weeks after the first dose of the anti-CTLA4 antagonist antibody.

7. The method of any one of claims 1-6, wherein the method comprises administering more doses of the anti-ICOS agonist antibody than the anti-CTLA4 antagonist antibody.

8. The method of any one of claims 1-7, wherein the method comprises administering four doses of the anti-CTLA4 antagonist antibody.

9. The method of any one of claims 1-8, wherein the method comprises administering at least one, at least two, at least three, at least four, or at least five doses of the anti-ICOS agonist antibody after the last dose of the anti-CTLA4 antagonist antibody has been administered.

10. The method of any one of claims 1-9, wherein the method comprises:
a) administering a first dose of the anti-CTLA4 antagonist antibody,
b) administering a first dose of the anti-ICOS agonist antibody three weeks after the first dose of the anti-CTLA4 antagonist antibody,
c) administering a second dose of the anti-CTLA4 antagonist antibody three weeks after the first dose of the anti-ICOS agonist antibody,
d) administering a second dose of the anti-ICOS agonist antibody three weeks after the second dose of the anti-CTLA4 antagonist antibody,
e) administering a third dose of the anti-CTLA4 antagonist antibody three weeks after the second dose of the anti-ICOS agonist antibody,
f) administering a third dose of the anti-ICOS agonist antibody three weeks after the third dose of the anti-CTLA4 antagonist antibody,
g) administering a fourth dose of the anti-CTLA4 antagonist antibody three weeks after the third dose of the anti-ICOS agonist antibody, and
h) administering a fourth dose of the anti-ICOS agonist antibody three weeks after the fourth dose of the anti-CTLA4 antagonist antibody.

11. The method of claim 10, comprising administering a fifth dose of the anti-ICOS agonist antibody six weeks after the fourth dose of the anti-ICOS agonist antibody.

12. The method of claim 11, comprising administering a sixth dose of the anti-ICOS agonist antibody six weeks after the fifth dose of the anti-ICOS agonist antibody.

13. The method of any one of claims 1-12, wherein each dose of the anti-ICOS agonist antibody is 0.1 mg/kg.

14. The method of any one of claims 1-12, wherein each dose of the anti-ICOS agonist antibody is 0.03 mg/kg.

15. The method of any one of claims 1-14, wherein the anti-CTLA4 antagonist antibody is selected from ipilimumab, tremelimumab, AGEN1181 (Agenus), AGEN1884 (Agenus), AGEN2041 (Agenus), and IBI310 (Innovent Biologics).

16. The method of claim 15, wherein the anti-CTLA4 antagonist antibody is ipilimumab.

17. The method of any one of claims 1-16, wherein each dose of the anti-CTLA4 antagonist antibody is 3 mg/kg.

18. The method of any one of claims 1-17, wherein administration of the first dose of the anti-CTLA4 antagonist antibody results in the emergence or increase of an ICOS^{hi} T-cell population in the peripheral blood of the subject prior to the administration of said anti-ICOS agonist antibody.

19. The method of any one of claims 1-18, wherein the anti-ICOS agonist antibody is selected from vopratelimab, GSK-3359069 (GSK), KY1044 (Kymab), KY1055 (Kymab), and BMS-986226 (Bristol-Myers Squibb).

20. The method of any one of claims 1-18, wherein the anti-ICOS agonist antibody comprises an HCDR1 comprising the amino acid sequence of SEQ ID NO: 9; an HCDR2 comprising the amino acid sequence of SEQ ID NO: 10; an HCDR3 comprising the amino acid sequence of SEQ ID NO: 11; an LCDR1 comprising the amino acid sequence of SEQ ID NO: 12; an LCDR2 comprising the amino acid sequence of SEQ ID NO: 13; and an LCDR3 comprising the amino acid sequence of SEQ ID NO: 14.

21. The method of claim 20, wherein the anti-ICOS agonist antibody comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 7 and the VL is at least 90%, 91%, 92%, 93%, 94%, 95%, 96%, 97%, 98%, 99%, or 100% identical to the amino acid sequence of SEQ ID NO: 8.

22. The method of any one of claims 1-18, wherein the anti-ICOS agonist antibody comprising a VH comprising the amino acid sequence of SEQ ID NO: 7 and a VL comprises the amino acid sequence of SEQ ID NO: 8.

23. The method of any one of claims 1-18, wherein the anti-ICOS agonist antibody comprises a heavy chain comprising the amino acid sequence of SEQ ID NO: 15 and a light chain comprising the amino acid sequence of SEQ ID NO: 16.

24. The method of any one of claims 1-18, wherein the anti-ICOS agonist antibody comprises a heavy chain comprising the amino acid sequence of SEQ ID NO: 17 and a light chain comprising the amino acid sequence of SEQ ID NO: 16.

25. The method of any one of claims 1-24, wherein the subject has a cancer selected from melanoma, lung cancer, non-small cell lung cancer (NSCLC), small cell lung cancer (SCLC), urethral cancer, renal cell carcinoma (RCC) (e.g., clear cell RCC), gastric cancer, bladder cancer, endometrial cancer, MSI-H cancer of any organ, diffuse large B-cell lymphoma (DLBCL), Hodgkin's lymphoma, ovarian cancer (e.g., endometrioid ovarian cancer), head & neck squamous cell cancer (HNSCC), acute myeloid leukemia (AML), rectal cancer, refractory testicular cancer, small bowel cancer, metastatic cutaneous squamous cell cancer, cervical cancer, MSI-high colon cancer, esophageal cancer, mesothelioma, breast cancer, and triple negative breast cancer (TNBC).

26. The method of claim 25, wherein the cancer is selected from lung cancer, non-small cell lung cancer (NSCLC), and small cell lung cancer (SCLC).

27. The method of claim 26, wherein the cancer is urethral cancer.

28. The method of any one of claims 1-27, wherein the subject has not previously been treated with PD-1 therapy.

29. The method of any one of claims 1-28, wherein the subject has previously been treated with at least one dose or cycle of PD-1 therapy.

30. The method of claim 29, wherein the subject showed, as a best overall response (BOR) to the PD-1 therapy, stable disease, partial response, or complete response.

31. The method of any one of claims 28-30, wherein the PD-1 therapy is PD-1 specific or PD-L1 specific.

32. The method of any one of claims 28-31, wherein the PD-1 therapy is an anti-PD-1 antibody or an anti-PD-L1 antibody.
